(19) United States
(12) Patent Application Publication
(10) Pub. No.: US 2005/0004161 A1
(43) Pub. Date: Jan. 6, 2005

(54) 4-oxo-4,7-dihydrothieno[2,3-b]pyridine-5-carboxamides as antiviral agents

(75) Inventors: Mark Edward Schnute, O'Fallon, MO (US); David John Anderson, Kalamazoo, MI (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/881,960

(22) Filed: Jun. 29, 2004

Related U.S. Application Data
(60) Provisional application No. 60/484,336, filed on Jul. 2, 2003.

(51) Int. Cl. C07D 498/02; A61K 31/4743

(52) U.S. Cl. 514/301; 546/114

(57) ABSTRACT

The invention provides a compound of formula I: 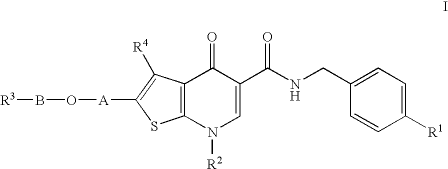
wherein A, B, R¹, R², R³, and R⁴ are as defined in the specification. The compounds of the present invention are useful for treating viral infections, in particular a herpesviral infection.

This application claims the benefit of U. S. Provisional application Ser. No. 60/484,336, filed Jul. 2, 2003, the contents of which is hereby incorporated by reference in it's entirety.

### FIELD OF THE INVENTION

The present invention provides 4-oxo-4,7-dihydrothieno[2,3-b]pyridine-5-carboxamides that are useful as antivirals, for example, as agents against viruses of the herpes family.

### BACKGROUND OF THE INVENTION

The herpesviruses comprise a large family of double stranded DNA viruses. They are also a source of the most common viral illnesses in man. Eight of the herpes viruses, herpes simplex virus types 1 and 2 (HSV-1 and HSV-2), varicella zoster virus (VZV), human cytomegalovirus (HCMV), Epstein-Barr virus (EBV), and human herpes viruses 6,7, and 8 (HHV-6, HHV-7, and HHV-8), have been shown to infect humans.

HSV-1 and HSV-2 cause herpetic lesions on the lips and genitals, respectively. They also occasionally cause infections of the eye and encephalitis. HCMV causes birth defects in infants and a variety of diseases in immunocompromised patients such as retinitis, pneumonia, and gastrointestinal disease. VZV is the causative agent of chicken pox and shingles. EBV causes infectious mononucleosis. It can also cause lymphomas in immunocompromised patients and has been associated with Burkitt's lymphoma, nasopharyngeal carcinoma, and Hodgkins disease. HHV-6 is the causative agent of roseola and may be associated with multiple sclerosis and chronic fatigue syndrome. HHV-7 disease association is unclear, but it may be involved in some cases of roseola. HHV-8 has been associated with Karposi's sarcoma, body cavity based lymphomas, and multiple myeloma.

Infection by reactivation of herpesviruses is associated with several cardiovascular diseases or conditions in the host such as atherosclerosis and restenosis resulting in inflammation of coronary vessel walls. It is thought that in many patients suffering from restenosis following coronary atherectomy viral infection particularly by CMV plays an important role in the proliferation of the disease. Atherosclerosis is believed to be associated with the overall infectious disease burden in the host and particularly by the herpesviruses such as HSV, CMV, and EBV.

Infection in the animal population (livestock and companion) by strains of herpesviruses is endemic including cattle (Bovine herpesvirus 1-5, BHV), sheep (Ovine herpesvirus 1 and 2), dog (Canine herpesvirus 1), horse (Equine herpesvirus 1-8, EHV), cat (Feline herpesvirus 1, FHV), swine (pseudorabies virus, PRV), and many species of fowl. In the case of bovine herpesvirus infection, animals may suffer from ocular, respiratory, or digestive disorders. Pseudorabies is an extremely contagious viral pathogen infecting several species such as cattle, horses, dogs, cats, sheep, and goats leading to rapid death. The virus is benign in adult swine, however, it remains contagious and leads to high mortality in pigs under three weeks. Infection of horses by equine herpesvirus may lead to neurological syndromes, respiratory disease, and neonatal disease. Herpesvirus infection in cats leads to the disease known as feline viral rhinotracheitis (FVR) which is characterized by rhinitis, tracheitis, laryngitis, and conjunctivitis.

### INFORMATION DISCLOSURE

U.S. Pat. No. 6,239,142 (Schnute et al.) describes 4-oxo-4,7-dihydrothieno[2,3-b]pyridine-5-carboxamides which are useful as antiviral agents. In particular the compounds can be used to treat or prevent herpes viral infections.

EP-A-443568 refers to fused thiophene derivatives, which are said to have angiotensin II antagonist activity and antihypertensive activity. In one embodiment, the thiophene ring can be fused to a pyridine ring having a group containing at least two phenyl rings attached to the nitrogen atom.

WO95/18405 discloses further bicyclic thiophene derivatives. The compounds are said to be effective as gonadotropin releasing hormone antagonists. It is among others suggested to employ the compounds as agents for the prevention or treatment of several hormone dependent diseases and also as fertility controlling agents.

### SUMMARY OF THE INVENTION

The present invention relates to a compound of formula I: 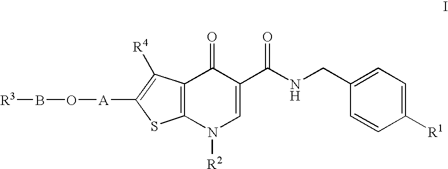
or a pharmaceutically acceptable salt thereof wherein
A is C₁₋₇ alkyl;
B is C₂₋₇ alkyl optionally substituted by one or more R⁵;
R¹ is
   (a) Cl,
   (b) Br,
   (c) F,
   (d) CN, or
   (e) NO₂;
R² is
   (a) (CH₂CH₂O)ᵢR⁶,
   (b) het, wherein said het is bonded via a carbon atom,
   (c) aryl,
   (d) C₁₋₇alkyl which may be partially unsaturated and is optionally substituted by one or more R⁷ substituents, or
   (e) C₃₋₈cycloalkyl which may be partially unsaturated and optionally substituted by one or more substituents selected from a group consisting of R⁷ or C₁₋₇alkyl optionally substituted by R⁷;
R³ is
   (a) aryl,
   (b) het,
   (c) OR⁸,
   (d) SR⁸,
   (e) NR⁸R⁹,
   (f) CH═N(OC₁₋₇alkyl), or
   (g) ON═C(R⁶)₂;
R⁴ is
   (a) H,
   (b) halo, or
   (c) C₁₋₄alkyl optionally substituted by halo;
R⁵ is
   (a) OR⁶,
   (b) SR⁶,
   (c) NR⁹R⁹,
   (d) halo,
   (e) oxo, or
   (f) phenyl optionally substituted by halo, C₁₋₇alkyl or C₁₋₇alkoxy,
R⁶ is
   (a) H, or
   (b) C₁₋₇alkyl;
R⁷ is
   (a) OR¹⁰,
   (b) SR¹⁰,
   (c) NR⁹R⁹,
   (d) NR⁹(COR¹¹)
   (e) halo,
   (f) CONHR¹¹,
   (g) CONR¹¹R¹¹,
   (h) CO₂H,
   (i) CO₂R¹¹,
   (j) het,
   (k) aryl,
   (l) cyano,
   (m) oxo, or
   (n) SOₘR¹¹;
R⁸ is
   (a) aryl, or
   (b) het;
R⁹ is
   (a) H,
   (b) phenyl,
   (c) C₃₋₈cycloalkyl, or
   (d) C₁₋₁₆alkyl optionally substituted by OH, phenyl, pyridinyl, or halo;
R¹⁰ is
   (a) H,
   (b) aryl,
   (c) het, wherein said het is bonded through a carbon atom,
   (d) C₁₋₇alkyl optionally substituted by aryl, het, OR⁶, SR⁶, NR⁶R⁶, halo, or C₃₋₈cycloalkyl optionally substituted by OR⁶, or
   (e) C₃₋₈cycloalkyl optionally substituted by one or more substituents selected from halo, OR⁶ SR⁶, or NR⁶R⁶,
R¹¹ is
   (a) aryl,
   (b) het,
   (c) C₃₋₈cycloalkyl, or
   (d) C₁₋₇alkyl which may be partially unsaturated and is optionally substituted by one or more substituents selected from a group consisting of NR⁶R⁶, OR⁶, SR⁶, halo, het, or aryl;
each i is independently 2, 3, or 4;
each m is independently 1 or 2;
aryl is a phenyl radical optionally fused to a carbocyclic radical wherein aryl is optionally substituted with one or more R¹² substituents or any two adjacent R¹² substituents taken together constitute a group of the formula —O(CH₂)ₘO—;
het is a 4-16 membered saturated or unsaturated monocyclic, bicyclic, or tricyclic ring system having one (1), two (2), three (3) or four (4) heteroatoms selected from the group consisting of oxygen (—O—), sulfur (—S—), oxygenated sulfur such as sulfinyl (S═O) and sulfonyl ( S(═O)₂), or nitrogen, or an N-oxide thereof wherein any het is optionally substituted with one or more oxo (═O) or R¹² substituents;
R¹² is
   (a) halo,
   (b) OR¹³,
   (c) SR⁶,
   (d) NR⁶R⁶,
   (e) phenyl, optionally substituted by halo, C₁₋₇alkyl, or C₁₋₇alkoxy,
   (f) cyano,
   (g) nitro,
   (h) CONR⁶R⁶,
   (i) CO₂R⁶,
   (j) SO₂NR⁶R⁶,
   (k) NR⁶(COR⁶),
   (l) C₁₋₇alkyl which is optionally partially unsaturated and is optionally substituted by one or more R¹⁴, or
   (m) C₃₋₈cycloalkyl;
R¹³ is
   (a) H
   (b) C₁₋₄alkyl optionally substituted by fluoro,
   (c) phenyl optionally substituted by halo, C₁₋₇alkyl, or C₁₋₇alkoxy, or
   (d) —(CH₂CH₂O)ₘR⁶;
R¹⁴ is
   (a) phenyl, optionally substituted by halo, C₁₋₇alkyl, or C₁₋₇alkoxy,
   (b) OR⁶,
   (c) SR⁶,
   (d) NR⁶R⁶,
   (e) 4-morpholine,
   (f) CO₂R⁶,
   (g) CONR⁶R⁶, or
   (h) halo.

The present invention further provides:
a pharmaceutical composition comprising a compound of formula I or a pharmaceutically acceptable salt thereof, and a pharmaceutically acceptable carrier;
a method of treating or preventing a herpesviral infection comprising administering to a mammal in need of such treatment, a compound of formula I or a pharmaceutically acceptable salt thereof, wherein the method is administered orally, parenterally, topically, rectally, nasally, sublingually or transdermally; and
a method for the treatment of atherosclerosis and restenosis comprising administering to a mammal in need of such treatment, a compound of formula I or a pharmaceutically acceptable salt thereof, wherein the method is administered orally, parenterally, topically, rectally, nasally, sublingually or transdermally;

### DETAILED DESCRIPTION OF THE INVENTION

The following definitions are used, unless otherwise described. Halo denotes fluoro, chloro, bromo, or iodo. Alkyl, alkoxy, etc. denote both straight and branched groups; but reference to an individual radical such as “propyl” embraces only the straight chain radical, a branched chain isomer such as “isopropyl” being specifically referred to. When alkyl can be partially unsaturated, the alkyl chain may comprise one or more (e.g. 1, 2, 3, or 4) double or triple bonds in the chain.

The term “aryl” refers to a phenyl radical optionally fused to a carbocyclic radical such as a benzene ring.

The term “het” refers to a het is a 4-16 membered saturated or unsaturated monocyclic, bicyclic, or tricyclic ring system having one (1), two (2), three (3) or four (4) heteroatoms selected from the group consisting of oxygen (—O—), sulfur (—S—), oxygenated sulfur such as sulfinyl (S═O) and sulfonyl (S(═O)₂), or nitrogen, or an N-oxide thereof. Preferbably, het is a 5-, or 6-membered saturated or unsaturated heterocyclic ring having 1, 2, or 3 heteroatoms selected from the group consisting of oxygen, sulfur, and nitrogen optionally fused to a benzene ring. “Het” may be bonded via heterocyclic moiety, or via the fused benzene ring moiety.

“Partially unsaturated”, for example, a C₁₋₇alkyl which is optionally partially unsaturated, means the named substitutent has one or more unsaturations, such as one or more double bonds, one or more triple bonds, or both.

“Optional” or “optionally” mean that the subsequently described event or condition may but need not occur, and that the description includes instances where the event or condition occurs and instances in which it does not. For example, “optionally substituted” means that the named substituent may be present but need not be present, and the description includes situations where the named substituent is included and situations where the named substituent is not included.

“Mammal” denotes humans and animals. Animals specifically refer to, for example, food animals or companion animals.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antiviral activity using the standard tests described herein, or using other similar tests which are well known in the art.

The carbon atom content of various hydrocarbon-containing moieties is indicated by a prefix designating a lower and upper number of carbon atoms in the moiety, i.e., the prefix Cᵢ₋ⱼ indicates a moiety of the integer “i” to the integer “j” carbon atoms, inclusive. Thus, for example, C₁₋₇alkyl refers to alkyl of one to seven carbon atoms, inclusive.

The compounds of the present invention are generally named according to the IUPAC or CAS nomenclature system. Abbreviations that are well known to one of ordinary skill in the art may be used (e.g. “Ph” for phenyl, “Me” for methyl, “Et” for ethyl, “h” for hour or hours, “rt” for room temperature, and “rac” for racemic mixture).

Specifically, C₁₋₇ alkyl can be methyl, ethyl, propyl, isopropyl, butyl, iso-butyl, sec-butyl, pentyl, 3-pentyl, hexyl, or heptyl; C₃₋₈cycloalkyl can be cyclopropyl, cyclobutyl, cyclopentyl, cyclohexyl, cycloheptyl, or cyclooctyl; C₁₋₇alkoxy can be methoxy, ethoxy, propoxy, isopropoxy, butoxy, iso-butoxy, sec-butoxy, pentoxy, 3-pentoxy, hexyloxy, 1-methylhexyloxy, or heptyloxy.

When C₁₋₇ alkyl is partially unsaturated, it can specifically be vinyl, allyl, 1-propenyl, 2-propenyl, 1-butenyl, 2-butenyl, 3-butenyl, 1,3-butadienyl, 1-pentenyl, 2-pentenyl, 3-pentenyl, 4-pentenyl 1-hexenyl, 2-hexenyl, 3-hexenyl, 4-hexenyl, 5-hexenyl, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, or 5-hexynyl.

Specific examples of “het” are, but not limited to, pyridyl, pyrimidinyl, pyridazinyl, pyrazinyl, pyranyl, imidazolyl, imidazolinyl, imidazolidinyl, pyrazolyl, pyrazolidinyl, oxazolyl, oxathiazolyl, oxadiazolyl, thiazolyl, isothiazole, furanyl, thienyl, pyrrolyl, isopyrrolyl, thiadiazolyl, thiatriazole, triazolyl, tetrazolyl, thiatriazolyl, phthalimide, benzofuranyl, benzothienyl, benzoxazolyl, indolyl, benzothiazolyl, furo[2,3-b]pyridinyl, furo[2,3-c]pyridinyl, furo[3,2-c]pyridinyl, furo[3,2-b]pyridinyl, triazinyl, quinolinyl, isoquinolinyl, quinazolinyl, quinoxalinyl, cinnolinyl, benzotriazinyl, purinyl, benzimidazolyl, azetidinyl, pyrrolidinyl, isoxazolidinyl, piperidinyl, piperazinyl, morpholinyl, oxazinanyl, azepanyl, oxazepane, dioxanyl, oxathianyl, hydantoinyl, or azabicyclo[2.2.1]heptyl. Each of these moieties may be substituted as appropriate or can include their corresponding N-oxides were appropriate.

If the compounds of the present invention contain a chiral element, the said compounds can be present as enantiomers or diastereomers, and the invention covers both racemates as well as the enantiomerically enriched compounds. Furthermore, all tautomeric forms of the instant compounds are within the scope of the invention.

The compounds of the present invention can also be in the form of their pharmaceutically acceptable salts or derivatives.

A specific value for R¹ is F, Cl, or Br.

A more specific value for R¹ is Cl.

A specific value for R² is C₁₋₇alkyl which may be partially unsaturated and is optionally substituted by one or more substituents selected from a group consisting of R⁷.

A more specific value for R² is C₁₋₇alkyl.

A more specific value for R² is methyl.

A more specific value for R² is ethyl.

A more specific value for R² is C₁₋₇alkyl substituted by one or more aryl or het.

A more specific value for R² is benzyl, 3-phenylpropyl, 2-morpholinoethyl, 3-morpholinopropyl, 2-piperidin-1-ylethyl, 3-piperidin-1-ylpropyl, 2-(1-methylpyrrolidin-2-yl)ethyl, or 2-pyrrolidin-1-ylethyl.

A more specific value for R² is C₁₋₇alkyl substituted with one or more hydroxy.

A more specific value for R² is 2-hydroxyethyl, 3-hydroxypropyl, 2,3-dihydroxypropyl, or 5-hydroxypentyl.

A more specific value for R² is C₁₋₇alkyl substituted by C₁₋₇alkoxy.

A more specific value for R² is 2-methoxyethyl.

A more specific value for R² is C₁₋₄alkyl substituted by NR⁹R⁹.

A more specific value for R² is 2-(diethylamino)ethyl or 2-(dimethylamino)ethyl.

A specific value for R² is C₃₋₈cycloalkyl which may be partially unsaturated and optionally substituted by one or more substituents selected from a group consisting of R⁷ or C₁₋₇alkyl optionally substituted by R⁷;

A more specific value for R² is cyclopropyl, 2-phenylcyclopropyl, 2-fluorocyclopropyl, cyclobutyl, or cyclohexyl.

A specific value for R² is het, wherein said het is bound via a carbon atom.

A more specific value for R² is pyridin-2-yl, 6-methylpyridin-2-yl, 4,6-dimethylpyridin-2-yl, pyridin-3-yl, pyridin-4-yl, pyrazin-2-yl, 5-ethyl-1,3,4-thiadiazol-2-yl, 5-cyclopropyl-1,3,4-thiadiazol-2-yl, 1,3-thiazol-2-yl, 5-methyl-1,3-thiazol-2-yl, 4-methyl-1,3-thiazol-2-yl, isoxazol-3-yl, or 3-methylisoxazol-5-yl.

A specific value for R² is aryl.

A more specific value for R² is phenyl.

A specific value for R³ is aryl.

A more specific value for R³ is phenyl, 1,1′-biphenyl-4-yl, 2-chlorophenyl, 3-chlorophenyl, 4-chlorophenyl, 2-methoxyphenyl, 3-methoxyphenyl, 4-methoxyphenyl, 2-methylphenyl, 4-methylphenyl, 4-bromophenyl, 2-naphthyl, 3,5-bis(trifluoromethyl)phenyl, 4-fluorophenyl, 4-(trifluoromethyl)phenyl, 3-cyanophenyl, 4-cyanophenyl, 3-(aminocarbonyl)phenyl, 4-(dimethylamino)phenyl, or 3-bromo-4-methoxyphenyl.

A more specific value for R³ is phenyl.

A specific value for R³ is het.

A more specific value for R³ is pyridin-4-yl, pyridin-3-yl, pyridin-2-yl, or 2-furyl.

A specific value for R³ is OR⁸.

A more specific value for R³ is phenoxy, 2-methylphenoxy, 2-methoxyphenoxy, 2-chlorophenoxy, 4-chlorophenoxy, 1-naphthyloxy, 3-nitrophenoxy, 2,3-dimethoxyphenoxy, 4-methoxyphenoxy, 4-fluorophenoxy, quinolin-8-yloxy, pyridin-2-yloxy, pyridin-4-yloxy, pyrimidin-2-yloxy, pyrazin-2-yloxy, 1,3-thiazol-2-yloxy, 1,3-thiazol-4-yloxy, and 1,3-thiazol-5-yloxy.

A specific value for R³ is SR⁸.

A more specific value for R³ is phenylthio.

A specific value for R³ is NR⁸R⁹.

A more specific value for R³ is NR⁸R⁹ wherein R⁸ is phenyl and R⁹ is methyl or ethyl.

A specific value for R³ is ((1-methylethylidene)amino)oxy.

A specific value for R³ is ethoxyimino.

A specific value for R⁴ is H or C₁₋₄alkyl.

A more specific value for R⁴ is H.

A specific value for A is C₁₋₃alkyl.

A more specific value for A is CH₂.

A specific value for B is C₂₋₄alkyl, optionally substituted by R⁵.

A more specific value for B is C₂₋₄alkyl optionally substituted by R⁵wherein R⁹ is OH, OC₁₋₄alkyl, oxo, or NR⁹R⁹ wherein R⁹ is independently H or C₁₋₁₆alkyl.

A more specific value for B is —CH₂CH(OH)—, —CH₂CH(═O)—, or —CH₂CH(NHC₁₋₁₆alkyl)-.

A more specific compound of formula I is a compound wherein any aryl or het is optionally substituted with one or two substituents selected from the group consisting of halo, hydroxy, C₁₋₇alkoxy, trifluoromethoxy, (C₁₋₃alkyl)₂N, phenyl, cyano, C₁₋₇ alkyl, or trifluoromethyl.

A more specific compound of formula I is a compound of formula IA:

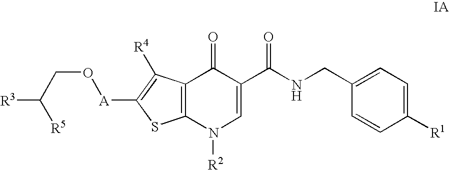

A more specific compound of formula I is a compound of formula IB:

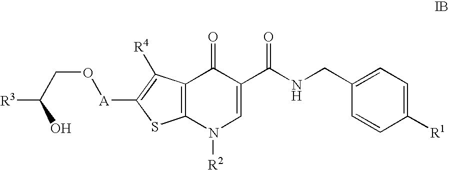

Examples of the present invention are:
(1) N-(4-chlorobenzyl)-2-((((2S)-2-hydroxy-2-phenylethyl)oxy)methyl)-7-methyl-4-oxo-4,7-dihydrothieno[2,3-b]pyridine-5-carboxamide;
(2) 2-((((2S)-2-(1,1′-biphenyl-4-yl)-2-hydroxyethyl)oxy)methyl)-N-(4-chlorobenzyl)-7-methyl-4-oxo-4,7-dihydrothieno[2,3-b]pyridine-5-carboxamide;
(3) rac-N-(4-chlorobenzyl)-2-((2-hydroxy-3-(((1-methylethylidene)amino)-oxy)propoxy)methyl)-7-methyl-4-oxo-4,7-dihydrothieno[2,3-b]pyridine-5-carboxamide;
(4) rac-N-(4-chlorobenzyl)-2-(((3-(ethoxyimino)-2-hydroxypropyl)oxy)methyl)-7-methyl-4-oxo-4,7-dihydrothieno[2,3-b]pyridine-5-carboxamide;
(5) rac-N-(4-chlorobenzyl)-2-((2-hydroxy-2-phenylethoxy)methyl)-7-methyl-4-oxo-4,7-dihydrothieno[2,3-b]pyridine-5-carboxamide
(6) N-(4-chlorobenzyl)-2-((((2S)-2-(2-chlorophenyl)-2-hydroxyethyl)oxy)methyl)-7-methyl-4-oxo-4,7-dihydrothieno[2,3-b]pyridine-5-carboxamide;
(7) N-(4-chlorobenzyl)-2-((((2S)-2-hydroxy-2-(2-methoxyphenyl)ethyl)oxy)methyl)-7-methyl-4-oxo-4,7-dihydrothieno[2,3-b]pyridine-5-carboxamide;
(8) N-(4-chlorobenzyl)-2-((((2S)-2-hydroxy-2-(3-methoxyphenyl)ethyl)oxy)methyl)-7-methyl-4-oxo-4,7-dihydrothieno[2,3-b]pyridine-5-carboxamide;
(9) N-(4-chlorobenzyl)-2-((((2S)-2-hydroxy-2-(2-methylphenyl)ethyl)oxy)methyl)-7-methyl-4-oxo-4,7-dihydrothieno[2,3-b]pyridine-5-carboxamide;
(10) N-(4-chlorobenzyl)-2-((((2S)-2-hydroxy-2-(4-methylphenyl)ethyl)oxy)methyl)-7-methyl-4-oxo-4,7-dihydrothieno[2,3-b]pyridine-5-carboxamide;
(11) N-(4-chlorobenzyl)-2-((((2S)-2-hydroxy-2-(4-bromophenyl)ethyl)oxy)methyl)-7-methyl-4-oxo-4,7-dihydrothieno[2,3-b]pyridine-5-carboxamide;
(12) N-(4-chlorobenzyl)-2-((((2S)-2-hydroxy-2-(2-naphthyl)ethyl)oxy)methyl)-7-methyl-4-oxo-4,7-dihydrothieno[2,3-b]pyridine-5-carboxamide;
(13) 2-((((2S)-2-((3,5-bis(trifluoromethyl)phenyl)-2-hydroxyethyl)oxy)methyl)-N-(4-chlorobenzyl)-7-methyl-4-oxo-4,7-dihydrothieno[2,3-b]pyridine-5-carboxamide;
(14) N-(4-chlorobenzyl)-2-((((2S)-2-(3-chlorophenyl)-2-hydroxyethyl)oxy)methyl)-7-methyl-4-oxo-4,7-dihydrothieno[2,3-b]pyridine-5-carboxamide;
(15) N-(4-chlorobenzyl)-2-((((2S)-2-(4-chlorophenyl)-2-hydroxyethyl)oxy)methyl)-7-methyl-4-oxo-4,7-dihydrothieno[2,3-b]pyridine-5-carboxamide;
(16) N-(4-chlorobenzyl)-2-((((2S)-2-(4-fluorophenyl)-2-hydroxyethyl)oxy)methyl)-7-methyl-4-oxo-4,7-dihydrothieno[2,3-b]pyridine-5-carboxamide;
(17) N-(4-chlorobenzyl)-2-((((2S)-2-hydroxy-2-(4-(trifluoromethyl)phenyl)-ethyl)oxy)methyl)-7-methyl-4-oxo-4,7-dihydrothieno[2,3-b]pyridine-5-carboxamide;
(18) N-(4-chlorobenzyl)-2-((((2S)-2-hydroxy-2-(4-methoxyphenyl)ethyl)oxy)methyl)-7-methyl-4-oxo-4-7-dihydrothieno[2,3-b]pyridine-5-carboxamide;
(19) N-(4-chlorobenzyl)-2-((((2S)-2-(3-cyanophenyl)-2-hydroxyethyl)oxy)methyl)-7-methyl-4-oxo-4,7-dihydrothieno[2,3-b]pyridine-5-carboxamide;
(20) 2-((((2S)-2-(3-(aminocarbonyl)phenyl)-2-hydroxyethyl)oxy)methyl)-N-(4-chlorobenzyl)-7-methyl-4-oxo-4,7-dihydrothieno[2,3-b]pyridine-5-carboxamide;
(21) methyl 4-((1S)-2-((5-(((4-chlorobenzyl)amino)carbonyl)-7-methyl-4-oxo-4,7-dihydrothieno[2,3-b]pyridin-2-yl)methoxy)-1 -hydroxyethyl)benzoate;
(22) N-(4-chlorobenzyl)-2-((((2S)-2-(4-(dimethylamino)phenyl)-2-hydroxyethyl)oxy)methyl)-7-methyl-4-oxo-4,7-dihydrothieno[2,3-b]pyridine-5-carboxamide;
(23) 2-((((2S)-2-(3-bromo-4-methoxyphenyl)-2-hydroxyethyl)oxy)methyl)-N-(4-chlorobenzyl)-7-methyl-4-oxo-4,7-dihydrothieno[2,3-b]pyridine-5-carboxamide;
(24) N-(4-chlorobenzyl)-2-((((2S)-2-hydroxy-2-pyridin-2-ylethyl)oxy)methyl)-7-methyl-4-oxo-4,7-dihydrothieno[2,3-b]pyridine-5-carboxamide;
(25) N-(4-chlorobenzyl)-2-((((2S)-2-hydroxy-2-pyridin-4-ylethyl)oxy)methyl)-7-methyl-4-oxo-4,7-dihydrothieno[2,3-b]pyridine-5-carboxamide;
(26) N-(4-chlorobenzyl)-2-((((2R)-2-(2-furyl)-2-hydroxyethyl)oxy)methyl)-7-methyl-4-oxo-4,7-dihydrothieno[2,3-b]pyridine-5-carboxamide;
(27) N-(4-chlorobenzyl)-2-((((2S)-2-hydroxy-2-phenylpropyl)oxy)methyl)-7-methyl-4-oxo-4,7-dihydrothieno[2,3-b]pyridine-5-carboxamide;
(28) N-(4-chlorobenzyl)-2-((((2S)-2-hydroxy-2-phenylbutyl)oxy)methyl)-7-methyl-4-oxo-4,7-dihydrothieno[2,3-b]pyridine-5-carboxamide;
(29) N-(4-chlorobenzyl)-2-((((2S)-2-hydroxy-3-methyl-2-phenylbutyl)oxy)methyl)-7-methyl-4-oxo-4,7-dihydrothieno[2,3-b]pyridine-5-carboxamide;
(30) N-(4-chlorobenzyl)-2-((((2S)-2-hydroxy-2,3-diphenylproply)oxy)methyl)-7-methyl-4-oxo-4,7-dihydrothieno[2,3-b]pyridine-5-carboxamide;
(31) rac-N-(4-chlorobenzyl)-2-(((2-hydroxy-3-methoxy-2-phenylpropyl)oxy)methyl)-7-methyl-4-oxo-4,7-dihydrothieno[2,3-b]pyridine-5-carboxamide;
(32) rac-N-(4-chlorobenzyl)-2-(((2-(4-fluorophenyl)-2-hydroxypropyl)oxy)methyl)-7-methyl-4-oxo-4,7-dihydrothieno[2,3-b]pyridine-5-carboxamide;
(33) rac-N-(4-chlorobenzyl)-2-(((2-(4-chlorophenyl)-2-hydroxypropyl)oxy)methyl)-7-methyl-4-oxo-4,7-dihydrothieno[2,3-b]pyridine-5-carboxamide;
(34) rac-2-(((2-(4-bromophenyl)-2-hydroxypropyl)oxy)methyl)-N-(4-chlorobenzyl)-7-methyl-4-oxo-4,7-dihydrothieno[2,3-b]pyridine-5-carboxamide;
(35) rac-N-(4-chlorobenzyl)-2-(((2-(4-cyanophenyl)-2-hydroxypropyl)oxy)methyl)-7-methyl-4-oxo-4,7-dihydrothieno[2,3-b]pyridine-5-carboxamide;
(36) N-(4-chlorobenzyl)-2-((((1R,2S)-2-hydroxy-1-methyl-2-phenylethyl)oxy)methyl)-7-methyl-4-oxo-4,7-dihydrothieno[2,3-b]pyridine-5-carboxamide;
(37) N-(4-chlorobenzyl)-2-((((2R)-2-hydroxy-3-phenoxypropyl)oxy)methyl)-7-methyl-4-oxo-4,7-dihydrothieno[2,3-b]pyridine-5-carboxamide;
(38) N-(4-chlorobenzyl)-2-((((2R)-2-hydroxy-3-(2-methylphenoxy)propyl)oxy)methyl)-7-methyl-4-oxo-4,7-dihydrothieno[2,3-b]pyridine-5-carboxamide;
(39) N-(4-chlorobenzyl)-2-((((2R)-2-hydroxy-3-(2-methoxyphenoxy)propyl)oxy)methyl)-7-methyl-4-oxo-4,7-dihydrothieno[2,3-b]pyridine-5-carboxamide;
(40) N-(4-chlorobenzyl)-2-((((2R)-3-(4-chlorophenoxy)-2-hydroxypropyl)oxy)methyl)-7-methyl-4-oxo-4,7-dihydrothieno[2,3-b]pyridine-5-carboxamide;
(41) N-(4-chlorobenzyl)-2-((((2R)-2-hydroxy-3-(1-naphthyloxy)propyl)oxy)methyl)-7-methyl-4-oxo-4,7-dihydrothieno[2,3-b]pyridine-5-carboxamide;
(42) N-(4-chlorobenzyl)-2-((((2R)-3-(2-chlorophenoxy)-2-hydroxypropyl)oxy)methyl)-7-methyl-4-oxo-4,7-dihydrothieno[2,3-b]pyridine-5-carboxamide;
(43) N-(4-chlorobenzyl)-2-((((2R)-2-hydroxy-3-(3-nitrophenoxy)propyl)oxy)methyl)-7-methyl-4-oxo-4,7-dihydrothieno[2,3-b]pyridine-5-carboxamide;
(44) N-(4-chlorobenzyl)-2-((((2R)-3-(2,3-dimethoxyphenoxy)-2-hydroxypropyl)oxy)methyl)-7-methyl-4-oxo-4,7-dihydrothieno[2,3-b]pyridine-5-carboxamide;
(45) N-(4-chlorobenzyl)-2-((((2R)-2-hydroxy-3-(4-methoxyphenoxy)propyl)oxy)methyl)-7-methyl-4-oxo-4,7-dihydrothieno[2,3-b]pyridine-5-carboxamide;
(46) N-(4-chlorobenzyl)-2-((((2R)-3-(4-fluorophenoxy)-2-hydroxypropyl)oxy)methyl)-7-methyl-4-oxo-4,7-dihydrothieno[2,3-b]pyridine-5-carboxamide;
(47) rac-N-(4-chlorobenzyl)-2-(((2-hydroxy-3-(quinolin-8-yloxy)propyl)oxy)methyl)-7-methyl-4-oxo-4,7-dihydrothieno[2,3-b]pyridine-5-carboxamide;
(48) N-(4-chlorobenzyl)-2-((((2R)-2-hydroxy-3-(pyridin-2-yloxy)propyl)oxy)methyl)-7-methyl-4-oxo-4,7-dihydrothieno[2,3-b]pyridine-5-carboxamide;
(49) N-(4-chlorobenzyl)-2-((((2R)-2-hydroxy-3-(pyridin-4-yloxy)propyl)oxy)methyl)-7-methyl-4-oxo-4,7-dihydrothieno[2,3-b]pyridine-5-carboxamide;
(50) N-(4-chlorobenzyl)-2-((((2R)-2-hydroxy-3-(pyridin-2-yloxy)propyl)oxy)methyl-7-methyl-4-oxo-4,7-dihydrothieno[2,3-b]pyridine-5-carboxamide;
(51) N-(4-chlorobenzyl)-2-((((2R)-2-hydroxy-3-(pyrazin-2-yloxy)propyl)oxy)methyl)-7-methyl-4-oxo-4,7-dihydrothieno[2,3-b]pyridine-5-carboxamide;
(52) N-(4-chlorobenzyl)-2-((((2R)-2-hydroxy-3-(1,3-thiazol-2-yloxy)propyl)oxy)methyl)-7-methyl-4-oxo-4,7-dihydrothieno[2,3-b]pyridine-5-carboxamide;
(53) N-(4-chlorobenzyl)-2-((((2R)-2-hydroxy-3-(1,3-thiazol-4-yloxy)propyl)oxy)methyl)-7-methyl-4-oxo-4,7-dihydrothieno[2,3-b]pyridine-5-carboxamide;
(54) N-(4-chlorobenzyl)-2-((((2R)-2-hydroxy-3-(1,3-thiazol-5-yloxy)propyl)oxy)methyl)-7-methyl-4-oxo-4,7-dihydrothieno[2,3-b]pyridine-5-carboxamide;
(55) rac-N-(4-chlorobenzyl)-2-((2-hydroxy-3-(methyl(phenyl)amino)propoxy)methyl)-7-methyl-4-oxo-4,7-dihydrothieno[2,3-b]pyridine-5-carboxamide;
(56) rac-N-(4-chlorobenzyl)-2-((2-hydroxy-3-(ethyl(phenyl)amino)propoxy)methyl)-7-methyl-4-oxo-4,7-dihydrothieno[2,3-b]pyridine-5-carboxamide;
(57) N-(4-chlorobenzyl)-2-((((2R)-2-hydroxy-3-(phenylthio)propyl)oxy)methyl)-7-methyl-4-oxo-4,7-dihydrothieno[2,3-b]pyridine-5-carboxamide;
(58) N-(4-Chlorobenzyl)-2-((((3R)-3-hydroxy-3-phenylpropyl)oxy)methyl)-7-methyl-4-oxo-4,7-dihydrothieno[2,3-b]pyridine-5-carboxamide;
(59) rac-N-(4-chlorobenzyl)-2-((3-hydroxy-3-(3-methylphenyl)propoxy)methyl)-7-methyl-4-oxo-4,7-dihydrothieno[2,3-b]pyridine-5-carboxamide;
(60) rac-N-(4-chlorobenzyl)-2-((3-hydroxy-3-(3-methoxyphenyl)propoxy)methyl)-7-methyl-4-oxo-4,7-dihydrothieno[2,3-b]pyridine-5-carboxamide;
(61) rac-N-(4-chlorobenzyl)-2-((3-(3-chlorophenyl)-3-hydroxypropoxy)methyl)-7-methyl-4-oxo-4,7-dihydrothieno[2,3-b]pyridine-5-carboxamide;
(62) rac-2-((3-(3-bromophenyl)-3-hydroxypropoxy)methyl)-N-(4-chlorobenzyl)-7-methyl-4-oxo-4,7-dihydrothieno[2,3-b]pyridine-5-carboxamide;
(63) rac-N-(4-chlorobenzyl)-2-((3-(3-fluorophenyl)-3-hydroxypropoxy)methyl)-7-methyl-4-oxo-4,7-dihydrothieno[2,3-b]pyridine-5-carboxamide;
(64) rac-N-(4-chlorobenzyl)-2-((3-hydroxy-3-(4-methylphenyl)propoxy)methyl)-7-methyl-4-oxo-4,7-dihydrothieno[2,3-b]pyridine-5-carboxamide;
(65) rac-N-(4-chlorobenzyl)-2-((3-hydroxy-3-(4-methoxyphenyl)propoxy)methyl)-7-methyl-4-oxo-4,7-dihydrothieno[2,3-b]pyridine-5-carboxamide;
(66) rac-2-((3-(4-bromophenyl)-3-hydroxypropoxy)methyl)-N-(4-chlorobenzyl)-7-methyl-4-oxo-4,7-dihydrothieno[2,3-b]pyridine-5-carboxamide;
(67) rac-N-(4-chlorobenzyl)-2-((3-(3,4-dimethoxyphenyl)-3-hydroxypropoxy)methyl)-7-methyl-4-oxo-4,7-dihydrothieno[2,3-b]pyridine-5-carboxamide;
(68) N-(4-chlorobenzyl)-7-ethyl-2-((((2S)-2-hydroxy-2-phenylethyl)oxy)methyl)-4-oxo-4,7-dihydrothieno[2,3-b]pyridine-5-carboxamide;
(69) N-(4-chlorobenzyl)-2-((((2S)-2-hydroxy-2-phenylethyl)oxy)methyl)-4-oxo-7-propyl-4,7-dihydrothieno[2,3-b]pyridine-5-carboxamide;
(70) N-(4-chlorobenzyl)-2-((((2S)-2-hydroxy-2-phenylethyl)oxy)methyl)-7-(2-methoxy-ethyl)-4-oxo-4,7-dihydrothieno[2,3-b]pyridine-5-carboxamide;
(71) N-(4-chlorobenzyl)-7-(2-hydroxyethyl)-2-((((2S)-2-hydroxy-2-phenylethyl)oxy)methyl)-4-oxo-4,7-dihydrothieno[2,3-b]pyridine-5-carboxamide;
(72) N-(4-chlorobenzyl)-2-((((2S)-2-hydroxy-2-phenylethyl]oxy)methyl)-7-(2-morpholin-4-ylethyl)4-oxo-4,7-dihydrothieno[2,3-b]pyridine-5-carboxamide;
(73) N-(4-chlorobenzyl)-3,7-dimethyl-2-((((2S)-2-hydroxy-2-phenylethyl)oxy)methyl)-4-oxo-4,7-dihydrothieno[2,3-b]pyridine-5-carboxamide;
(74) N-(4-chlorobenzyl)-7-methyl-4-oxo-2-((2-oxo-2-phenylethoxy)methyl)-4,7-dihydrothieno[2,3-b]pyridine-5-carboxamide;
(75) N-(4-chlorobenzyl)-2-((2-(ethylamino)-2-phenylethoxy)methyl)-7-methyl-4-oxo-4,7-dihydrothieno[2,3-b]pyridine-5-carboxamide;
(76) N-(4-chlorobenzyl)-7-methyl-4-oxo-2-((2-phenyl-2-(propylamino)ethoxy)methyl)-4,7-dihydrothieno[2,3-b]pyridine-5-carboxamide;
(77) N-(4-chlorobenzyl)-2-((2-(dodecylamino)-2-phenylethoxy)methyl)-7-methyl-4-oxo-4,7-dihydrothieno[2,3-b]pyridine-5-carboxamide;
(78) N-(4-chlorobenzyl)-2-((2-(cyclopropylamino)-2-phenylethoxy)methyl)-7-methyl-4-oxo-4,7-dihydrothieno[2,3-b]pyridine-5-carboxamide;
(79) N-(4-chlorobenzyl)-7-methyl-4-oxo-2-((2-phenyl-2-((pyridin-2-ylmethyl)amino)ethoxy)methyl)-4,7-dihydrothieno[2,3-b]pyridine-5-carboxamide;
(80) rac-N-(4-Chlorobenzyl)-2-((2-hydroxy-1-phenylethoxy)methyl)-7-methyl-4-oxo-4,7-dihydrothieno[2,3-b]pyridine-5-carboxamide;
or a pharmaceutically acceptable salt thereof.

The compounds of the invention can be prepared according to the following Charts A-H. All of the variables used in the scheme are as defined above or as in the claims.

Specific compounds of the present invention can be prepared as described in Chart A derived from a suitably substituted alkanol A.1. Alkanols of the formula A.1 can be prepared by procedures described in U.S. Pat. No. 6,239,142, which is incorporated herein by reference in its entirety, or by procedures described herein below. Reactions of alkanols A.1 with a suitably substituted epoxide in the presence of a strong base such as a hydride in a polar solvent (e.g. DMF) affords compounds of the formula A.2 wherein at least one of the R⁵ substituents to B is OH. It would be understood by those skilled in the art that in some cases transient protection of hydroxyl and other Lewis basic or acidic functionality present in the epoxide component or the alkanol component may be required to facilitate the coupling described in Chart A for which procedures are well established (Greene, T. W.; Wuts, P. G. M. Protective Groups in Organic Synthesis, 1999).

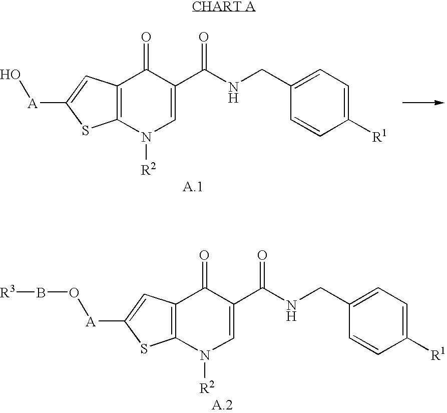

Epoxides employed in Chart A may be commercially available (e.g. (S)-styrene oxide) or prepared by methods commonly known to those skilled in the art of organic synthesis. Additional representative epoxides which can be employed include but are not limited to (S)-2-(4-methylphenyl)oxirane, (S)-2-(4-chlorophenyl)oxirane, (S)-2-(3-chlorophenyl)oxirane, (S)-2-(2-chlorophenyl)oxirane, and (S)-2-methyl-2-phenyloxirane (Spelberg, J. H. L.; Rink, R.; Kellog, R. M.; Janssen, D. B. Tetrahedron Asymmetry 1998, 9, 459-466); (S)-2-(4-trifluoromethylphenyl)oxirane and (S)-2-(3,5-ditrifluoromethylphenyl)oxirane (Weissman, S. A.; Rossen, K.; Reider, P. J. Org. Lett. 2001, 3,2513-2515); (S)-2-(2-methoxyphenyl)oxirane, (S)-2-(3-methoxyphenyl)oxirane, (S)-2-(4-methoxyphenyl)oxirane, (S)-2-(3-trifluoromethyl-phenyl)oxirane, (S)-2-(3-methylphenyl)oxirane, and (S)-5-oxirane-2-y-1,3-benzodioxole (Hamada, T.; Torii, T.; Izawa, K.; Noyori, R.; Ikariya, T. Org. Leff. 2002, 4,4373-4376); (S)-2-(1,1-biphen-4-yl)oxirane and (S)-2-(4-nitrophenyl)oxirane (Cho, B. T.; Yang, W. K.; Choi, O. K. J. Chem. Soc. Perkin Trans. 1 2001, 1204-1211); (S)-2-(4-cyanophenyl)oxirane (Pedragosa-Moreau, S.; Morisseau, C.; Zylber, J.; Archelas, A.; Baratti, J; Furstoss, R. J. Org. Chem. 1996, 61, 7402-7407); (S)-2-ethyl-2-phenyloxirane and (S)-2-benzyl-2-phenyloxirane (Capriati, V.; Florio, S.; Luisi, R.; Salomone, A. Org. Lett. 2002, 4, 2445-2448); 2-oxiran-2-ylpyridine, 3-oxiran-2-ylpyridine, and 4-oxiran-2-ylpyridine (Genzel, Y.; Archelas, A.; Broxterman, Q. B.; Schulze, B.; Furstoss, R. J. Org. Chem. 2001, 66, 538-543).

Compounds of the general formula A.1 are prepared according to procedures described in U.S. Pat. No. 6,239,142 or exemplified in Charts B-C below.

As described in Chart B, 3-bromo-2-chlorothiophene (B.1) is metalated with lithium diisopropyl amide in tetrahydrofuran at low temperature followed by addition to paraformaldehyde to provide alcohol B.2. The free hydroxyl is protected employing common methodology (Greene, T. W.; Wuts, P. G. M. Protective Groups in Organic Synthesis, 1999) such as the tert-butyldimethylsilyl ether (TBS) by treatment with the corresponding silyl chloride and a weak base (e.g. imidazole) in a polar solvent (e.g. DMF). Metalation of B.3 with n-butyl lithium followed by addition to N-methoxy-N-methylacetamide provides the methyl ketone B.4. Condensation of B.4 with diethyl carbonate in the presence of a strong base (e.g. sodium hydride) affords ketoester B.5. Compound B.5 is then refluxed in a mixture of acetic anhydride and triethylorthoformate to afford an intermediate enol ether which is then condensed with a primary amine or aniline (e.g. R²NH₂) to provide a compound of the formula B.6. The resulting enamines are cyclized by heating in the presence of a base (e.g. sodium hydride, potassium carbonate, or potassium tert-butoxide) in an appropriate solvent (e.g. THF, DMF, or tert-butanol) to provide B.7. Esters of the formula B.7 are converted to amides of the general formula B.8 by either (a) treatment with a substituted benzylamine (e.g. 4-chlorobenzylamine, 4-fluorobenzylamine, or 4-bromobenzylamine) at high temperature or (b) saponification by treatment with an inorganic base such as sodium hydroxide to afford the corresponding carboxylic acid which is then coupled with a substituted benzylamine mediated by 1,1′-carbonyidiimidazole (or other suitable carboxylic acid activating agent). Subsequent deprotection of the hydroxyl protecting group to afford A.1 (wherein A=CH₂) is accomplished through common procedures such as treatment with tetrabutylammonium fluoride in the case of silyl ether protection.

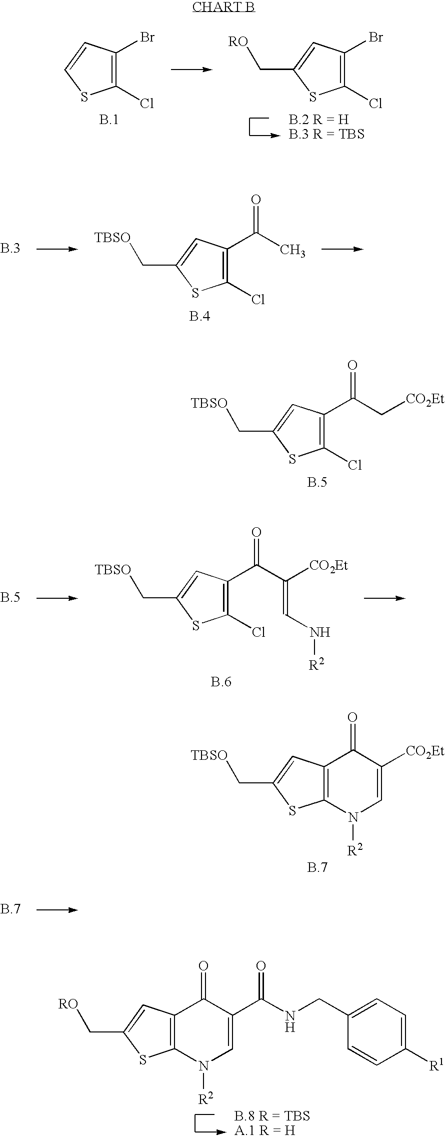

Alternatively, compounds of formula A.1 (wherein A=CH₂) may be prepared as described in Chart C. Ethyl 4-hydroxythieno[2,3-b]pyridine-5-carboxylate (J. Heterocyclic Chem. 1977, 14, 807) is metallated with from two to six equivalents of lithium diisopropylamide at low temperature and is then reacted with N,N-dimethylformamide to provide compound C.2. Treatment of C.2 with an appropriate reducing agent (e.g. NaBH₄) in a polar solvent (e.g. ethanol) affords the alcohol C.3. The resulting ester is then reacted with a substituted benzylamine (e.g. 4-chlorobenzylamine, 4-fluorobenzylamine, or 4-bromobenzylamine) at high temperature or under other common amide forming conditions well known to those skilled in the art to provide compounds of the formula C.4. Compound C.4 is alkylated at the ring nitrogen by treatment with an optionally substituted alkyl halide or alkyl sulfonate ester in the presence of a base (e.g. potassium carbonate) or by reaction with an optionally substituted alkanol under Mitsunobu conditions to afford compounds of the general formula A.1. Specific examples of such alkyl halides used in this reaction include but are not limited to iodomethane, iodoethane, 1-iodopropane, 1-iodobutane, and 1-bromo-2-methoxyethane. It would be understood by those skilled in the art that in some cases transient protection of hydroxyl functionality present in the R²X (X=halo or sulfonate) or R²OH reagent used in the above step may be required to facilitate the coupling described in Chart C or subsequent chemistry described in Chart A. Specific examples of such protected-hydroxyalkyl halides used in this reaction include but are not limited to 2-(2-bromoethoxy)tetrahydro-2H-pyran, 2-(3-bromopropoxy)tetrahydro-2H-pyran, 4-(bromomethyl)-2,2-dimethyl-1,3-dioxolane, 2-(2-(2-chloroethoxy)ethoxy)tetrahydro-2H-pyran, and 2-(chloromethoxy)ethyl benzoate. Procedures to deprotect these cases at the final or intermediate stage are well established (Greene, T. W.; Wuts, P. G. M. Protective Groups in Organic Synthesis, 1999).

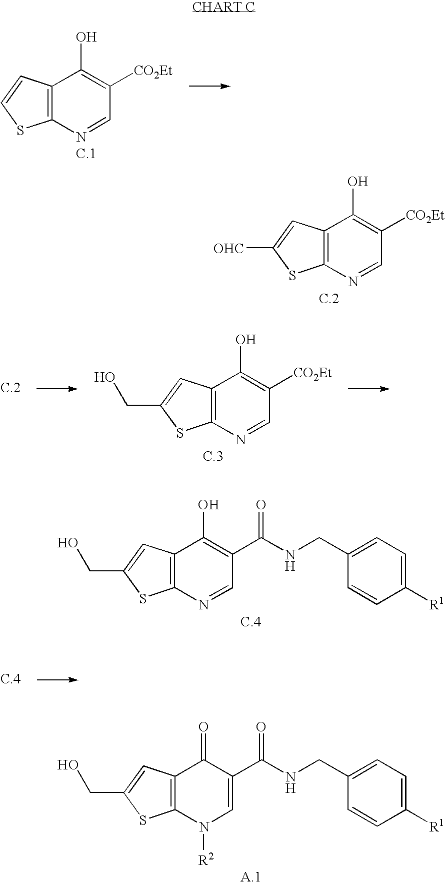

Compounds of formula I are also prepared according to Chart D. Employing methods described by Nagashima, N.; Ohno, M. Chem. Parm. Bull. 1991, 39, 1972-1982, an appropriately substituted ethane-1,2-diol or propane-1,3-diol is reacted with dibutyltin oxide followed by an alkylhalide of the formula D.1 (X═Cl, Br, or I) in the presence of cesium fluoride to afford compounds of the formula A.2 wherein at least one of the R⁵substituents to B is OH. It would be understood by those skilled in the art that in some cases transient protection of hydroxyl and other Lewis basic or acidic functionality present In the diol component or the alkyl halide component may be required to facilitate the coupling described in Chart D for which procedures are well established (Greene, T. W.; Wuts, P. G. M. Protective Groups in Organic Synthesis, 1999).

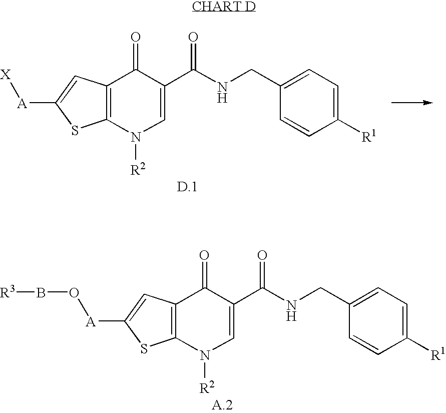

Ethane-1,2-diols employed in Chart D are prepared by methods commonly known to those skilled in the art of organic synthesis such as dihydroxylation of alkenes or hydrolysis of epoxides. Propane-1,3-diols are commonly prepared by reduction of 3-oxo-alkanoate esters by hydride reducing agents.

The precursors D.1 employed in Chart D may be prepared from the corresponding alkanols A.1 by halogenation under well known conditions (e.g. Lee reaction or reaction with oxalyl chloride), Chart E. Especially when A=CH₂, the corresponding alkanol is treated with methanesulfonyl chloride in the presence of an organic base (e.g. pyridine or 2,4,6-collidine) and if needed an activating agent (e.g. DMAP). Alkanols A.1 are prepared as described above.

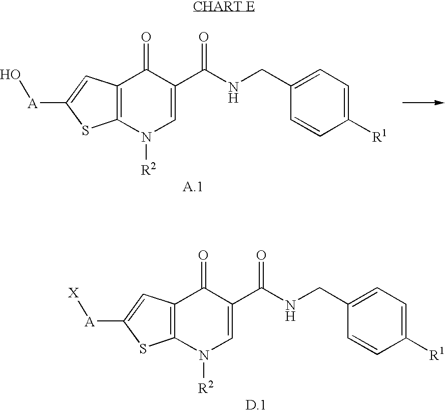

Alternatively, precursors D.1 employed in Chart D where A=CH₂ are prepared by treatment of a tertiary amine derivative F.1 (e.g. NR₂═N(CH₃)₂ or 4-morpholinyl) with ethylchloroformate in an appropriate solvent (e.g. chloroform, dichloromethane, 1,2-dichloroethane, or benzene), Chart F. Compounds of formula F.1 are prepared as described in U.S. Pat. No. 6,239,142 or as described in Chart G below.

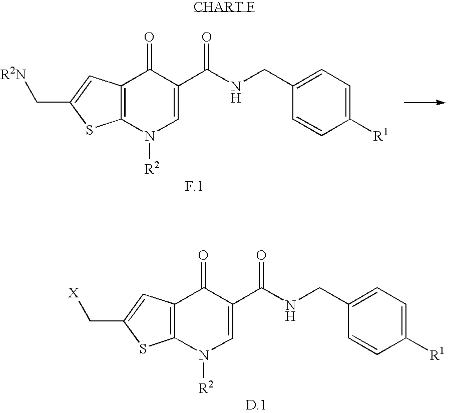

According to Chart G, 3-bromo-2-chlorothiophene (B.1) is metalated with lithium diisopropyl amide in tetrahydrofuran at low temperature and condensed with N,N-dimethylformamide to afford the carboxaldehyde G.1. Reductive amination of G.1 by treating with an amine (e.g. morpholine), acetic acid, and an appropriate reducing agent (e.g. sodium triacetoxyborohydride) affords thiophenes of the formula G.2. Metalation of G.2 with n-butyl lithium followed by addition to N-methoxy-N-methylacetamide provides the methyl ketone G.3. Condensation of G.3 with diethyl carbonate in the presence of a strong base (e.g. sodium hydride) affords ketoester G.4. The resulting ketoester is then treated with a benzylamine (e.g. 4-chlorobenzylamine, 4-fluorobenzylamine, or 4-bromobenzylamine) in refluxing xylene to provide ketoamides of the formula G.5. Compound G.5 is then refluxed in a mixture of acetic anhydride and triethylorthoformate to afford an intermediate enol ether which is then condensed with a primary amine or aniline (e.g. R²NH₂) to provide a compound of the 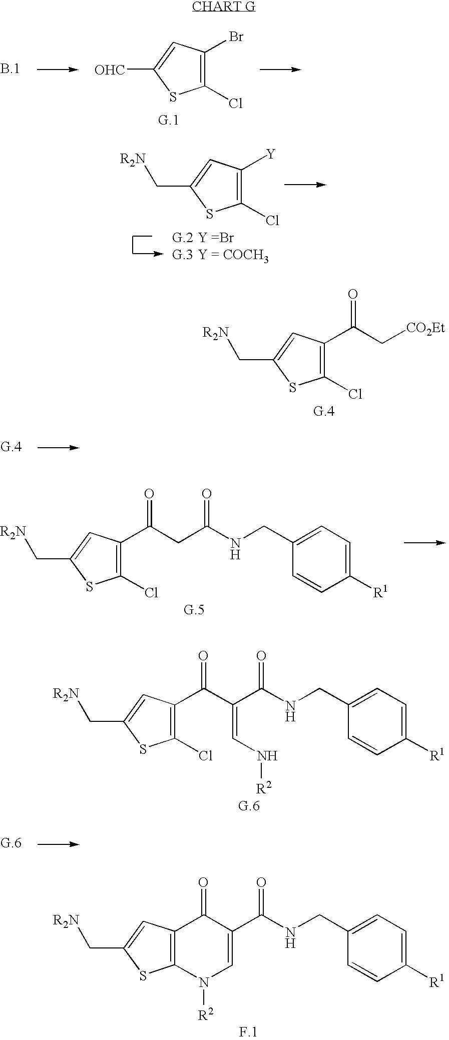
formula G.6. The resulting enamines are cyclized by heating in the presence of a base (e.g. sodium hydride, potassium carbonate, or potassium tert-butoxide) in an appropriate solvent (e.g. THF, DMF, or tert-butanol).

Specific examples of compounds of formula I wherein R⁵ is oxo or NR⁹R⁹ are prepared in analogy to the representative example shown in Chart H. Compounds of the formula H.1, prepared according to Chart D employing 1-phenylethane-1,2-diol, is oxidized with an appropriate oxidizing agent (e.g. Dess Martin periodinane) to afford ketones of the formula H.2. Subsequent treatment of H.2 with an amine of the formula HNR⁹R⁹ and an appropriate reducing agent (e.g. sodium cyanoborohydride) under reductive amination conditions affords compounds of the formula H.3.

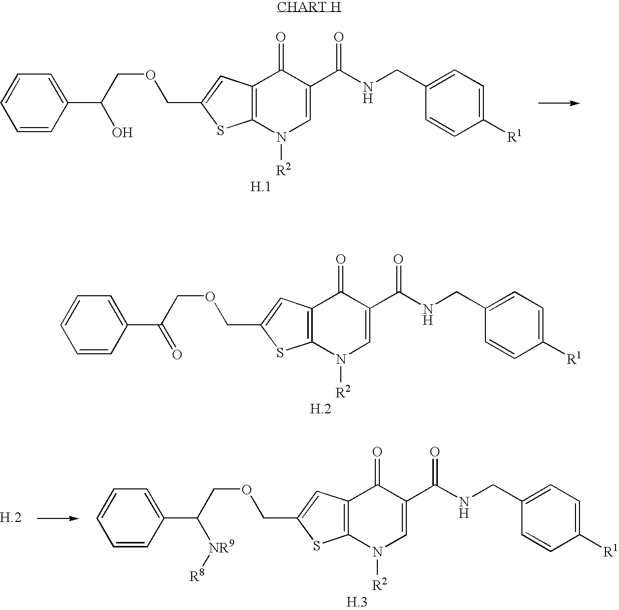

The compounds of Formula (I) may be prepared as single enantiomer or as a mixture of individual enantiomers which includes racemic mixtures. Methods to obtain preferentially a single enantiomer from a mixture of individual enantiomers or a racemic mixture are well known to those ordinarily skilled in the art of organic chemistry. Such methods include but are not limited to preferential crystallization of diastereomeric salts (e.g. tartrate or camphor sulfonate), covalent derivatization by a chiral, non-racemic reagent followed by separation of the resulting diastereomers by common methods (e.g. crystallization, chromatographic separation, or distillation) and chemical reversion to scalemic compound, Simulated Moving Bed technology, or high/medium-pressure liquid chromatography employing a chiral stationary phase (Eliel, E. L. Stereochemistry of Organic Compounds, 1994; Subramanian, G. Chiral Separation Techniques: A Practical Approach, 2001). These techniques may be performed on the final compounds of Formula (I) or on any intermediates to compounds of Formula (I) which bear a stereogenic center. Also, to facilitate separation by any of the methods described above, the compounds of Formula (I) or any intermediates to the compounds of Formula (I) which bear a stereogenic center may be transiently reacted with an achiral reagent, separated, and then reverted to scalemic compound by standard synthetic techniques.

The compounds of formula I can be used in the native form or as a salt. In cases where forming a stable nontoxic salt is desired, administration of the compound as a pharmaceutically acceptable salt may be appropriate. Examples of pharmaceutically acceptable salts are organic acid addition salts formed with acids which form a physiologically acceptable anion, for example, tosylate, methanesulfonate, acetate, citrate, malonate, tartarate, succinate, benzoate, ascorbate, ketoglutarate, glycerophosphate, and like salts. Suitable inorganic salts may also be formed, including hydrochloride, hydrobromide, sulfate, nitrate, bicarbonate, carbonate, and like salts. Alkali metal (for example, sodium, potassium or lithium) or alkaline earth metal (for example calcium) salts of carboxylic acids can also be made.

Pharmaceutically acceptable salts may be obtained using standard procedures well known in the art, for example, by reacting a sufficiently basic compound such as an amine with a suitable acid affording a physiologically acceptable anion.

Compounds of the present invention can conveniently be administered in a pharmaceutical composition containing the compound in combination with a suitable excipient, the composition being useful in combating viral infections. Pharmaceutical compositions containing a compound appropriate for antiviral use are prepared by methods and contain excipients which are well known in the art. A generally recognized compendium of such methods and ingredients is Remington's Pharmaceutical Sciences by E. W. Martin (Mark Publ. Co., 15th Ed., 1975). The compounds and compositions of the present invention can be administered parenterally, for example, by intravenous, intraperitoneal or intramuscular injection, topically, parenterally, orally, rectally, transmucosally, or intestinally depending on whether the preparation is used to treat internal or external viral infections. Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infusion techniques. Rectal administration includes suppositories. Transmucosal administration includes nasal aerosol or inhalation applications. Preferred routes of administration are oral and parenteral. Additionally, the compounds may be delivered using a sustained-release system. Various sustained-release materials have been established and are well known to those skilled in the art. Sustained-release capsules may, depending on their chemical nature, release the compounds for 24 hours or for up to several days. Additionally, the compounds may be delivered using a sustained-release system.

For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fuctose as a sweetening agent, methyl and propyl parabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid. Topical administrations include the treatment of infectious areas or organs readily accessible by local application, such as, for example, eyes, ears including external and middle ear infections, vaginal, open wound, or skin including the surface skin and the underneath dermal structures. It also includes transdermal delivery to generate a systemic effect.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157), and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The compound is conveniently administered in unit dosage form; for example, containing about 5 to about 1,000 mg, conveniently about 10 to about 750 mg, most conveniently about 50 to about 500 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

For internal infections, the compositions can be administered orally or parenterally at dose levels, calculated as the free base, of about 0.1 to about 300 mg/kg, preferably about 1.0 to about 30 mg/kg of mammal body weight, and can be used in man in a unit dosage form, administered one to four times daily in the amount of about 1 to about 1,000 mg per unit dose.

For parenteral administration or for administration as drops, as for eye infections, the compounds are presented in aqueous solution in a concentration of from about 0.1 to about 10%, more preferably about 0.1 to about 7%. The solution may contain other ingredients, such as emulsifiers, antioxidants or buffers.

Generally, the concentration of the compound(s) of formula I in a liquid composition, such as a lotion, will be from about 0.1 to about 25 wt-%, preferably from about 0.5 to about 10 weight percent. The concentration in a semi-solid or solid composition such as a gel or a powder will be about 0.1 to about 5 weight percent, preferably about 0.5 to about 2.5 weight percent.

The exact regimen for administration of the compounds and compositions disclosed herein will necessarily be dependent upon the needs of the individual subject being treated, the type of treatment and, of course, the judgment of the attending practitioner.

The antiviral activity of a compound of the invention can be determined using pharmacological models which are well known to the art, or using Test A described below.

The compounds of formula 1, pharmaceutically acceptable salts and derivatives thereof are useful as antiviral agents. Thus, they are useful to combat viral infections in mammals, including man and animals. The compounds are generally active against herpes viruses, and are particularly useful against the varicella zoster virus (VZV), the Epstein-Barr virus (EBV), the herpes simplex virus 1 and 2 (HSV-1 and HSV-2), the human herpes virus 6, 7, and 8 (HHV-6, HHV-7, and HHV-8) and the cytomegalovirus (CMV). In humans, these viruses lead to illnesses including herpes labialis and herpes genitalis (HSV), human cytomegalovirus (HCMV) disease in the immunocompromised, chickenpox and shingles (VZV), and mononucleosis and post-transplant lymphoproliferative disease (PTLD) (EBV). The compounds of the present invention may also be useful for the treatment of herpesviral infections in animals, for example, illnesses caused by bovine herpesvirus 1-5 (BHV), ovine herpesvirus 1 and 2, canine herpesvirus 1, equine herpesvirus 1-8 (EHV), feline herpesvirus 1 (FHV), and pseudorabies virus (PRV).

While many of the compounds of the present invention have shown activity against the CMV polymerase, these compounds may be active against the cytomegalovirus by this or other mechanisms of action. Thus, the description below of these compounds' activity against the CMV polymerase is not meant to limit the present invention to a specific mechanism of action.

Test A

The HCMV polymerase assay is performed using a scintillation proximity assay (SPA) as described in several references, such as N. D. Cook, et al., Pharmaceutical Manufacturing International, pages 49-53 (1992); K. Takeuchi, Laboratory Practice, September issue (1992); and U.S. Pat. No. 4,568,649 (1986), which are incorporated by reference herein. Reactions are performed in 96-well plates. The assay is conducted in 100 ul volume with 5.4 mM HEPES (pH 7.5), 11.7 mM KCl, 4.5 mM MgCl₂, 0.36 mg/ml BSA, and 90 nM ³H-dTTP. Assays are run with and without CHAPS, (3-[(3-cholamidopropyl)-dimethyl-ammonio]-1-propane-sulfonate) at a final concentration of 2 mM. HCMV polymerase is diluted in enzyme dilution buffer containing 50% glycerol, 250 mM NaCl, 10 mM HEPES (pH 7.5), 100 pg/mL BSA, and 0.01% sodium azide. The HCMV polymerase, which is expressed in recombinant baculovirus-infected SF-9 cells and purified according to literature procedures, is added at 10% (or 10 μL) of the final reaction volume, i.e., 100 μL. Compounds are diluted in 50% DMSO and 10 μL are added to each well. Control wells contain an equivalent concentration of DMSO. Unless noted otherwise, reactions are initiated via the addition of 6 nM biotinylated poly (dA)-oligo (dT) template/primer to reaction mixtures containing the enzyme, substrate, and compounds of interest. Plates are incubated in a 25° C. or 37° C. H₂O bath and terminated via the addition of 40 μL/reaction of 0.5 M EDTA (pH 8) per well. Reactions are terminated within the time-frame during which substrate incorporation is linear and varied depending upon the enzyme and conditions used, i.e., 30 min. for HCMV polymerase. Ten μL of streptavidin-SPA beads (20 mg/mL in PBS/10% glycerol) are added following termination of the reaction. Plates are incubated 10 min. at 37° C., then equilibrated to room temperature, and counted on a Packard Topcount. Linear regressions are performed and IC₅₀'s are calculated using computer software.

A modified version of the above HCMV polymerase assay is performed as described above, but with the following changes: Compounds are diluted in 100% DMSO until final dilution into assay buffer. In the previous assay, compounds are diluted in 50% DMSO. 4.5 mM Dithiothreitol (DTT) is added to the polymerase buffer. Also, a different lot of CMV polymerase is used, which appears to be more active resulting in a more rapid polymerase reaction. Representative compounds of formula I were found to be active in this assay.

The following examples are intended to illustrate the present invention. They should not be construed as limiting.

### EXAMPLES

### Example 1

### N-(4-Chlorobenzyl)-2-((((2S)-2-hydroxy-2-phenylethyl)oxy)methyl)-7-methyl-4-oxo-4,7-dihydrothieno]2,3-b]pyridine-5-carboxamide]

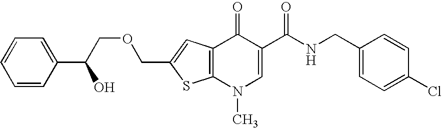

N-(4-Chlorobenzyl)-2-(hydroxymethyl)-7-methyl-4-oxo-4,7-dihydrothieno[2,3-b]pyridine-5-carboxamide (544 mg, prepared as described in U.S. Pat. No. 6,239,142) is suspended in DMF (25 mL) and sodium hydride (66 mg, 60% dispersion in mineral oil) is added. The mixture is heated to 40° C. and (S)-styrene oxide (190 μL) is added. The reaction mixture is then heated at 100° C. for 1 h and after cooling to room temperature is poured into water (250 mL). The mixture is extracted with EtOAc (4×100 mL). The combined organic layers are washed with water (100 mL) followed by brine (100 mL), dried (MgSO₄), and concentrated. The crude product is purified by column chromatography (CH₂Cl₂/methanol, 100/1; 50/1) to afford 42 mg of the title compound as a tan solid. Physical characteristics. M.p. 165-168° C.; ¹H NMR (300 MHz, DMSO-d₆) δ 10.55, 8.71, 7.40-7.21, 5.44, 4.75, 4.73, 4.53, 3.94, 3.54, 3.50. Rotation (25° C., D)=+14 (c 0.56, chloroform). Anal. Found: C, 62.02; H, 4.85; N, 5.72; Cl, 7.31; S, 6.52.

Employing procedures analogous to those described in Example 1 and utilizing the appropriate epoxide, Examples 2-4 are prepared. The corresponding epoxides are prepared according to literature procedures as follows, (S)-2-(1,1′-biphen-4-yl)oxirane (Example 2) (Cho, B. T.; Yang, W. K.; Choi, 0. K. J. Chem. Soc. Perkin Trans. 1 2001, 1204-1211); acetone O-(oxiran-2-yl)oxime (Example 3) (Leclerc, G. J. Med. Chem. 1980, 23, 620-624); and oxirane-2-carboxaldehyde O-ethyl oxime (Example 4) (Macchia, B.; Balsamo, A.; Breschi, M. C.; Chiellini, G.; Macchia, M.; Martinelli, A.; Martin, C.; Nardini, C.; Nencetti, S.; Rossello, A.; Scalizzi, R. J. Med. Chem. 1994, 37,1518-1525).

### Example 2

### 2-((((2S)-2-(1,1′-Biphenyl-4-yl)-2-hydroxyethyl)oxy)methyl)-N-(4-chlorobenzyl)-7-methyl-4-oxo-4,7-dihydrothieno[2,3-b]pyridine-5-carboxamide

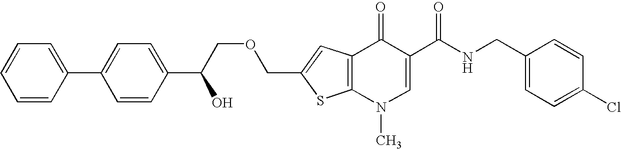

Physical characteristics. MS (El) m/z 558 (M⁺).

### Example 3

### rac-N-(4-Chlorobenzyl)-2-((2-hydroxy-3-(((1-methylethylidene)amino)oxy)propoxy)methyl)-7-methyl-4-oxo-4,7-dihydrothieno[2,3-b]pyridine-5-carboxamide

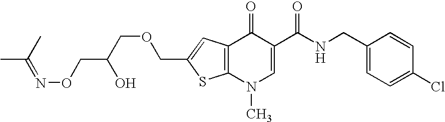

Physical characteristics. MS (El) m/z 491 (M⁺).

### Example 4

### rac-N-(4-Chlorobenzyl)-2-(((3-(ethoxyimino)-2-hydroxypropyl)oxy)methyl)-7-methyl-4-oxo-4,7-dihydrothieno[2,3-b]pyridine-5-carboxamide

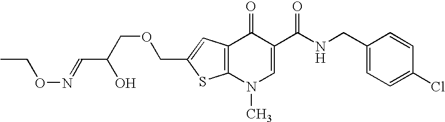

Physical characteristics. MS (El) m/z 477 (M⁺).

Preparation 1.

### N-(4-Chlorobenzyl)-2-(chloromethyl)-7-methyl-4-oxo-4,7-dihydrothieno[2,3-b]pyridine-5-carboxamide

Procedure A. N-(4-Chlorobenzyl)-2-(hydroxymethyl)-7-methyl-4-oxo-4,7-dihydrothieno[2,3-b]pyridine-5-carboxamide (3.00 g, prepared as described in U.S. Pat. No. 6,239,142) is dissolved in DMF (150 mL). DMAP (0.150 g), 2,4,6-collidine (2.73 mL), and methanesulfonyl chloride (1.60 mL) are added, and the reaction mixture is stirred at room temperature for 18 h. The reaction mixture is poured into water (300 mL). The resulting solid is filtered and triturated with acetonitrile to yield 2.75 g of the title compound. Physical characteristics. Mp 250-256° C. (dec); ¹H NMR (400 MHz, DMSO-d₆) δ 10.48, 8.74, 7.58, 7.41-7.33, 5.16, 4.55, 3.97; ¹³C NMR (DMSO-d₆) δ 172.5, 164.5, 151.8, 146.4, 138.9, 135.7, 131.7, 130.5, 129.5, 128.7, 124.0, 115.0, 43.4, 41.8, 41.1; MS (El) m/z 380 (M⁺); HRMS (FAB) m/z 381.0255 (M+H)⁺. Anal. Found: C, 53.34; H, 3.70; N, 7.30; Cl, 17.91; S, 8.51.

Procedure B. A 25 mL round-bottomed flask is charged with N-(4-chlorobenzyl)-7-methyl-2-(morpholin-4-ylmethyl)-4-oxo-4,7-dihydrothieno[2,3-b]pyridine-5-carboxamide (1.00 g, prepared as described in U.S. Pat. No. 6,239,142) and chloroform (10 ml) via syringe. Ethyl chloroformate (0.55 mL) is added via syringe with stirring under nitrogen. The slurry is heated to reflux overnight. Anhydrous diethyl ether (10 ml) is added to the slurry with stirring under nitrogen. The solid is filtered and washed with diethyl ether (3×10 mL). The product is dried in the vacuum oven at 40° C. to afford 0.93 g of the title compound as colorless crystals. Physical characteristics. ¹H NMR (400 MHz, TFA-d) δ 9.09, 7.69, 7.22, 4.81, 4.62, 4.27; ¹³C NMR (100 MHz, TFA-d) δ 167.6, 166.6, 156.3, 145.2, 143.6, 134.9, 133.3, 129.1, 129.0, 127.4, 119.6, 109.9, 45.2, 44.0, 38.0. Anal. Found: C, 53.44; H, 3.66; N, 7.35; Cl, 18.29.

### Example 5

### rac-N-(4-Chlorobenzyl)-2-((2-hydroxy-2-phenylethoxy)methyl)-7-methyl-4-oxo-4,7-dihydrothieno[2,3-b]pyridine-5-carboxamide

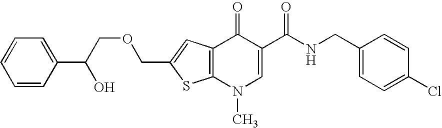

A mixture of rac-1-phenylethane-1,2-diol (705 mg) and dibutyltin oxide (1.27 g) in toluene (100 mL) is heated to reflux for 2 h with removal of water by a Dean-Stark trap. After cooling, the solvent is evaporated and the residue is treated with N-(4-chlorobenzyl)-2-(chloromethyl)-7-methyl-4-oxo-4,7-dihydrothieno[2,3-b]pyridine-5-carboxamide (Preparation 1, 2.9 g), cesium fluoride (1.55 g), and DMF (50 mL). The mixture is stirred under nitrogen at 55° C. for 18 h, and then the solvent is evaporated. The residue is purified by column chromatography (CHCl₃/methanol, 98/2) to afford 1.61 g of the title compound as a white solid. Physical characteristics. M.p. 181-182° C.; ¹H NMR (300 MHz, DMSO-d₆) δ 3.48-3.59, 3.95, 4.54, 4.69-4.78, 4.77, 5.45, 7.17-7.44, 8.73, 10.54. Anal. Found: C, 61.84; H, 4.76; N, 5.85.

Employing procedures analogous to those described in Example 5 and utilizing the appropriate ethane-1,2-diol, Examples 6-57 are prepared. The corresponding ethane-1,2-diols are prepared according to literature procedures as follows where not commercially available. (S)-1-(2-methoxyphenyl)ethane-1,2-diol (Example 7) as described in Wipf, P.; Hopkins, C. R. J. Org. Chem. 2001, 66,3133-3139. (S)-1-(3-methoxyphenyl)ethane-1,2-diol (Example 8) as described in Jones, G. B.; Guzel, M.; Heaton, S. B. Tetrahedon Asymmetry 2000, 11, 4303-4320. (S)-1-(2-methylphenyl)ethane-1,2-diol (Example 9), (S)-1-(4-methylphenyl)ethane-1,2-diol (Example 10), (S)-1-(4-bromophenyl)ethane-1,2-diol (Example 11), and (S)-1-(2-naphthyl)ethane-1,2-diol (Example 12) as described in Cho, B. T.; Chun, Y. S. J. Org. Chem. 1998, 63,5280-5282. (S)-1-(3,5-bis(trifluoromethyl)phenyl)ethane-1,2-diol (Example 13), (S)-1-(3-chlorophenyl)ethane-1,2-diol (Example 14), (S)-1-(4-chlorophenyl)ethane-1,2-diol (Example 15), (S)-1-(4-fluorophenyl)ethane-1,2-diol (Example 16), and (S)-1-(4-trifluoromethylphenyl)ethane-1,2-diol (Example 17) as described in Weissman, S. A.; Rossen, K.; Reider, P. J. Org. Lett. 2001, 3, 2513-2515. (S)-1-(4-methoxyphenyl)ethane-1,2-diol (Example 18) as described in Cho, B. T.; Chun, Y. S. Tetrahedron Asymmetry 1999, 10, 1843-1846. (S)-1-(3-cyanophenyl)ethane-1,2-diol (Example 19) and (S)-3-(1,2-dihydroxyethyl)benzamide (Example 20) as described in Watson, C. Y.; Whish, W. J. D.; Threadgill, M. D. Biorg. Med. Chem. 1998, 6, 721-734. Methyl (S)-4-(1,2-dihydroxyethyl)benzoate (Example 21) as described in Nymann, K.; Jensen, C.; Svendsen, J. S. Acta Chem. Scand. 1996, 50, 832-841. rac-1-(4-N,N-Dimethylaminophenyl)ethane-1,2-diol (Example 22) as described in lida, T.; Itaya, T. Tetrahedron 1993, 49, 10511-10530. rac-1-(3-Bromo-4-methoxyphenyl)ethane-1,2-diol (Example 23) as described in Lawrence, N. J.; Bushell, S. M. Tetrahedron Lett. 2001, 42, 7671-7674. (S)-1-Pyridin-2-ylethane-1,2-diol (Example 24) as described in Chelucci, G.; Cabras, M. A.; Saba, A. Tetrahedron Asymmetry 1994, 5, 1973-1978. (S)-1-Pyridin-4-ylethane-1,2-diol (Example 25) as described in Hauser, F. M.; Ellenberger, S. R.; Ellenberger, W. P. Tetrahedron Lett. 1988, 29, 4939-4942. (R)-1-(2-Furyl)ethane-1,2-diol (Example 26) as described in Simons, K. E.; Wang, G.; Heinz, T.; Giger, T.; Mallat, T.; Pfaltz, A.; Baiker, A. Tetrahedron Asymmetry 1995, 6, 505-518. (S)-2-Phenylpropane-1,2-diol (Example 27), (S)-2-phenylbutane-1,2-diol (Example 28), and (S)-3-methyl-2-phenylbutane-1,2-diol (Example 29) as described in Colunbo, L.; Giacomo, M. D.; Brusott, G.; Milano, E. Tetrahedron Lett. 1995, 36, 2863-2866. (S)-2,3-Diphenylpropane-1,2-diol (Example 30) as described in Hof, R. P; Kellog, R. M. Tetrahedron Asymmetry 1994, 5, 565-568. rac-3-Methoxy-2-phenylpropane-1,2-diol (Example 31) as described in Adam, W.; Treiber, A. J. Am. Chem. Soc. 1995,117,2686-2693. rac-2-(4-Fluorophenyl)propane-1,2-diol (Example 32), rac-2-(4-chlorophenyl)propane-1,2-diol (Example 33), rac-2-(4-bromophenyl)propane-1,2-diol (Example 34), and rac-2-(4-cyanophenyl)propane-1,2-diol (Example 35) as described in Cleij, M.; Archelas, A.; Furstoss, R. J. Org. Chem. 1999, 64, 5029-5035. (1S,2R)-1-Phenylpropane-1,2-diol (Example 36) as described in Takeshita, M.; Yaguchi, R.; Akutsu, N. Tetrahedron Asymmetry 1992, 3, 1369-1372. (R)-3-Phenoxypropane-1,2-diol (Example 37), (R)-3-(2-methylphenoxy)propane-1,2-diol (Example 38), (R)-3-(2-methoxyphenoxy)propane-1,2-diol (Example 39), and (R)-3-(4-chlorophenoxy)propane-1,2-diol (Example 40) as described in Kitaori, K.; Furukawa, Y.; Yoshimoto, H.; Otera, J. Tetrahedron 1999, 55, 14381-14390. (R)-3-(1-Naphthyloxy)propane-1,2-diol (Example 41), (R)-3-(2-chlorophenoxy)propane-1,2-diol (Example 42), and (R)-3-(3-nitrophenoxy)propane-1,2-diol (Example 43) as described in Egri, G.; Kolbert, A.; Balint, J.; Fogassy, E.; Novak, L.; Poppe, L. Tetrahedron Asymmetry 1998, 9, 271-283. (R)-3-(2,3-Dimethoxyphenoxy)propane-1,2-diol (Example 44) as described in Valoti, E.; Pallavicin, M, Villa, L.; Pezzetta, D. J. Org. Chem. 2001, 66, 1018-1025. (R)-3-(4Methoxyphenoxy)propane-1,2-diol (Example 45) as described in Takans, S.; Moriya, M.; Suzuki, M.; Iwabuchi, Y.; Sugihara, T.; Ogasawara, K. Heterocycles 1990, 31, 1555-1563. (R)-3-(4-Fluorophenoxy)propane-1,2-diol (Example 46) as described in Gurjar, M. K.; Sadalapure, K.; Adhikari, S.; Sarma, B.; Talukdar, A.; Chorghade, M. S. Heterocycles 1998, 48, 1471-1476. rac-3-(Quinolin-8-yloxy)propane-1,2-diol (Example 47) as described in Dishong, D. M.; Diamond, C. J.; Cinoman, M. I.; Gokel, G. W. J. Am. Chem. Soc. 1983, 105, 586-593. (R)-3-(Pyridin-2-yloxy)propane-1,2-diol (Example 48), (R)-3-(pyridin-4-yloxy)propane-1,2-diol (Example 49), (R)-3-(pyrimidin-2-yloxy)propane-1,2-diol (Example 50), (R)-3-(pyrazin-2-yloxy)propane-1,2diol (Example 51), (R)-3-(1,3-thiazol-2-yloxy)propane-1,2-diol (Example 52), (R)-3-(1,3-thiazol-4-yloxy)propane-1,2-diol (Example 53), and (R)-3-(1,3-thiazol-5-yloxy)propane-1,2-diol (Example 54) are prepared analogous to procedures described for the (S)-enantiomer in Barlow, J. J.; Block, M. H.; Hudson, J. A.; Leach, A.; Longridge, J. L.; Main, B. G.; Nicholson, S. J. Org. Chem. 1992, 57, 5158-5162 except (S)-2,2-dimethyl-1,3-dioxolane is employed. rac-3-(Methyl-(phenyl)amino)propane-1,2-diol (Example 55) as described in Davis, W.; Savige, W. E. J. Chem. Soc. 1950, 890-894. rac-3-(Ethyl(phenyl)amino)propane-1,2-diol (Example 56) as described in Malinovskii, M. S.; Perchick, V. W. Zhur. Obshchei Khim. 1957, 27, 1591-1593. (R)-3-(phenylthio)propane-1,2-diol (Example 57) as described in Yodo, M.; Matsushita, Y.; Ohsugi, E.; Harada, H. Chem. Pharm. Bull. 1988, 23, 620-624.

### Example 6

### N-(4-Chlorobenzyl)-2-((((2S)-2-(2-chlorophenyl)-2-hydroxyethyl)oxy)methyl)-7-methyl-4-oxo-4,7-dihydrothieno[2,3-b]pyridine-5-carboxamide

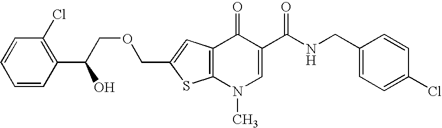

Physical characteristics. MS (El) m/z 516 (M⁺).

### Example 7

### N-(4-Chlorobenzyl)-2-((((2S)-2-hydroxy-2-(2-methoxyphenyl)ethyl)oxy)methyl)-7-methyl-4-oxo-4,7-dihydrothieno[2,3-b]pyridine-5-carboxamide

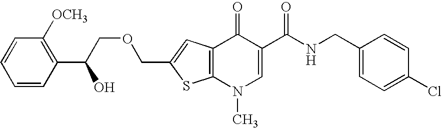

Physical characteristics. MS (El) m/z 512 (M⁺).

### Example 8

### N-(4-chlorobenzyl)-2-((((2S)-2-hydroxy-2-(3-methoxyphenyl)ethyl)oxy)methyl)-7-methyl-4-oxo-4,7-dihydrothieno[2,3-b]pyridine-5-carboxamide

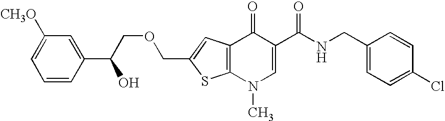

Physical characteristics. MS (El) m/z 512 (M⁺).

### Example 9

### N-(4-chlorobenzyl)-2-((((2S)-2-hydroxy-2-(2-methylphenyl)ethyl)oxy)methyl)-7-methyl-4-oxo-4,7-dihydrothieno[2,3-b]pyridine-5-carboxamide

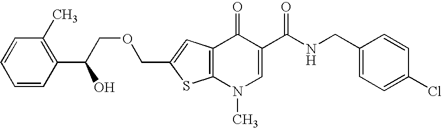

Physical characteristics. MS (El) m/z 496 (M⁺).

### Example 10

### N-(4-chlorobenzyl)-2-((((2S)-2-hydroxy-2-(4-methylphenyl)ethyl)oxy)methyl)-7-methyl-4-oxo-4,7-dihydrothieno[2,3-b]pyridine-5-carboxamide

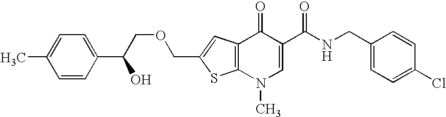

Physical characteristics. MS (El) m/z 496 (M⁺).

### Example 11

### N-(4-chlorobenzyl)-2-((((2S)-2-hydroxy-2-(4-bromophenyl)ethyl)oxy)methyl)-7-methyl-4-oxo-4,7-dihydrothieno[2,3-b]pyridine-5-carboxamide

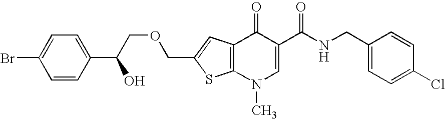

Physical characteristics. MS (El) m/z 560 (M⁺).

### Example 12

### N-(4-chlorobenzyl)-2-((((2S)-2-hydroxy-2-(2-naphthyl)ethyl)oxy)methyl)-7-methyl-4-oxo-4,7-dihydrothieno[2,3-b]pyridine-5-carboxamide

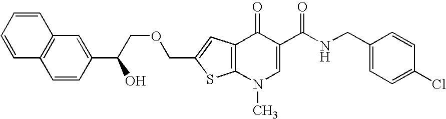

Physical characteristics. MS (El) m/z 532 (M⁺).

### Example 13

### 2-((((2S)-2-((3,5-bis(trifluoromethyl)phenyl)-2-hydroxyethyl)oxy)methyl)-N-(4-chlorobenzyl)-7-methyl-4-oxo-4,7-dihydrothieno[2,3-b]pyridine-5-carboxamide

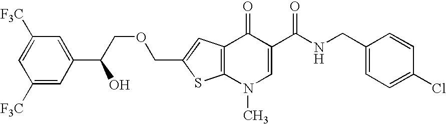

Physical characteristics. MS (El) m/z 618 (M⁺).

### Example 14

### N-(4-chlorobenzyl)-2-((((2S)-2-(3-chlorophenyl)-2-hydroxyethyl)oxy)methyl)-7-methyl-4-oxo-4,7-dihydrothieno[2,3-b]pyridine-5-carboxamide

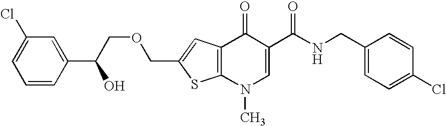

Physical characteristics. MS (El) m/z 516 (M⁺).

### Example 15

### N-(4-chlorobenzyl)-2-((((2S)-2-(4-chlorophenyl)-2-hydroxyethyl)oxy)methyl)-7-methyl-4-oxo-4,7-dihydrothieno[2,3-b]pyridine-5-carboxamide

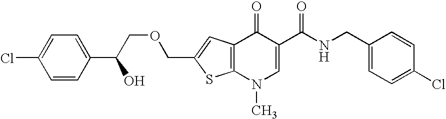

Physical characteristics. MS (El) m/z 516 (M⁺).

### Example 16

### N-(4-chlorobenzyl)-2-((((2S)-2-(4-fluorophenyl)-2-hydroxyethyl)oxy)methyl)-7-methyl-4-oxo-4,y-dihydrothieno[2,3-b]pyridine-5-carboxamide

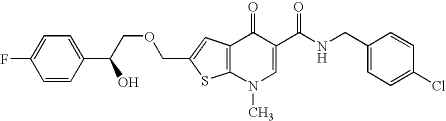

Physical characteristics. MS (El) m/z 500 (M⁺).

### Example 17

### N-(4-chlorobenzyl)-2-((((2S)-2-hydroxy-2-(4-(trifluoromethyl)phenyl)ethyl)oxy)methyl)-7-methyl-4-oxo-4,7-dihydrothieno[2,3-b]pyridine-5-carboxamide

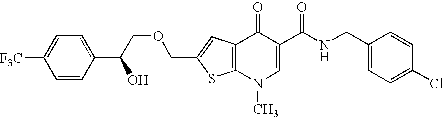

Physical characteristics. MS (El) m/z 550 (M⁺).

### Example 18

### N-(4-chlorobenzyl)-2-((((2S)-2-hydroxy-2-(4-methoxyphenyl)ethyl)oxy)methyl)-7-methyl-4-oxo-4,7-dihydrothieno[2,3-b]pyridine-5-carboxamide

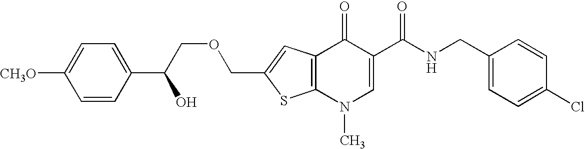

Physical characteristics. MS (El) m/z 512 (M⁺).

### Example 19

### N-(4-chlorobenzyl)-2-((((2S)-2-(3-cyanophenyl)-2-hydroxyethyl)oxy)methyl)-7-methyl-4-oxo-4,7-dihydrothieno[2,3-b]pyridine-5-carboxamide

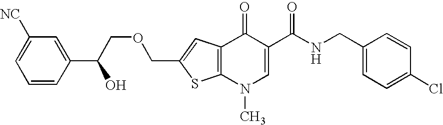

Physical characteristics. MS (El) m/z 507 (M⁺).

### Example 20

### 2-((((2S)-2-(3-(aminocarbonyl)phenyl)-2-hydroxyethyl)oxy)methyl)-N-(4-chlorobenzyl)-7-methyl-4-oxo-4,7-dihydrothieno[2,3-b]pyridine-5-carboxamide

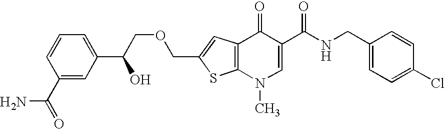

Physical characteristics. MS (El) m/z 525 (M⁺).

### Example 21

### methyl 4-((1S)-2-((5-(((4-chlorobenzyl)amino)carbonyl)-7-methyl-4-oxo-4,7-dihydrothieno[2,3-b]pyridin-2-yl)methoxy)-1-hydroxyethyl)benzoate

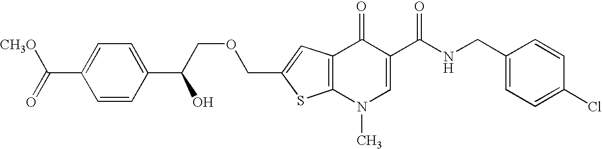

Physical characteristics. MS (El) m/z 540 (M⁺).

### Example 22

### N-(4-chlorobenzyl)-2-((((2S)-2-(4-(dimethylamino)phenyl)-2-hydroxyethyl)oxy)methyl)-7-methyl-4-oxo-4,7-dihydrothieno[2,3-b]pyridine-5-carboxamide

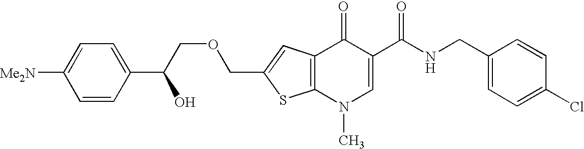

Physical characteristics. MS (El) m/z 525 (M⁺).

### Example 23

### 2-((((2S)-2-(3-bromo-4-methoxyphenyl)-2-hydroxyethyl)oxy)methyl)-N-(4-chlorobenzyl)-7-methyl-4-oxo-4,7-dihydrothieno[2,3-b]pyridine-5-carboxamide

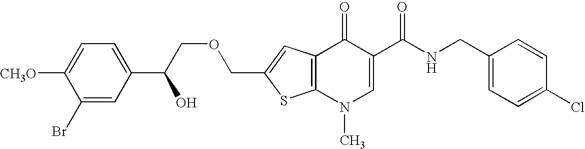

Physical characteristics. MS (El) m/z 590 (M⁺).

### Example 24

### N-(4-chlorobenzyl)-2-((((2S)-2-hydroxy-2-pyridin-2-ylethyl)oxy)methyl)-7-methyl-4-oxo-4,7-dihydrothieno[2,3-b]pyridine-5-carboxamide

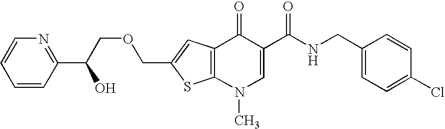

Physical characteristics. MS (El) m/z 483 (M⁺).

### Example 25

### N-(4-chlorobenzyl)-2-((((2S)-2-hydroxy-2-pyridin-4-ylethyl)oxy)methyl)-7-methyl-4-oxo-4,7-dihydrothieno[2,3-b]pyridine-5-carboxamide

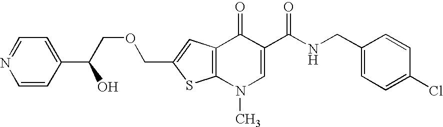

Physical characteristics. MS (El) m/z 483 (M⁺).

### Example 26

### N-(4-chlorobenzyl)-2-((((2R)-2-(2-furyl)-2-hydroxyethyl)oxy)methyl)-7-methyl-4-oxo-4,7-dihydrothieno[2,3-b]pyridine-5-carboxamide

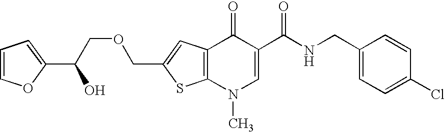

Physical characteristics. MS (El) m/z 472 (M⁺).

### Example 27

### N-(4-chlorobenzyl)-2-((((2S)-2-hydroxy-2-phenylpropyl)oxy)methyl)-7-methyl-4-oxo-4,7-dihydrothieno[2,3-b]pyridine-5-carboxamide

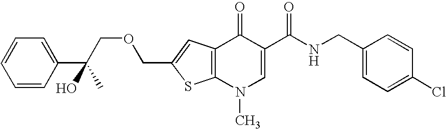

Physical characteristics. MS (El) m/z 496 (M⁺).

### Example 28

### N-(4-chlorobenzyl)-2-((((2S)-2-hydroxy-2-phenylbutyl)oxy)methyl)-7-methyl-4-oxo-4,7-dihydrothieno[2,3-b]pyridine-5-carboxamide

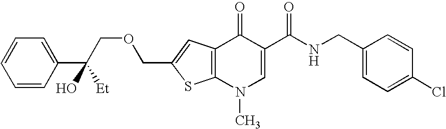

Physical characteristics. MS (El) m/z 510 (M⁺).

### Example 29

### N-(4-chlorobenzyl)-2-((((2S)-2-hydroxy-3-methyl-2-phenylbutyl)oxy)methyl)-7-methyl-4-oxo-4,7-dihydrothieno[2,3-b]pyridine-5-carboxamide

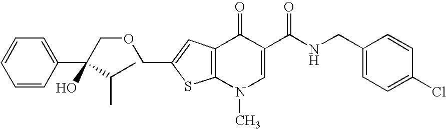

Physical characteristics. MS (El) m/z 524 (M⁺).

### Example 30

### N-(4-chlorobenzyl)-2-((((2S)-2-hydroxy-2,3-diphenylpropyl)oxy)methyl)-7-methyl-4-oxo-4,7-dihydrothieno[2,3-b]pyridine-5-carboxamide

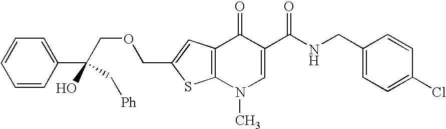

Physical characteristics. MS (El) m/z 572 (M⁺).

### Example 31

### rac-N-(4-chlorobenzyl)-2-(((2-hydroxy-3-methoxy-2-phenylpropyl)oxy)methyl)-7-methyl-4-oxo-4,7-dihydrothieno[2,3-b]pyridine-5-carboxamide

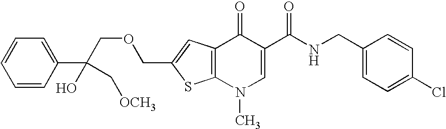

Physical characteristics. MS (El) m/z 526 (M⁺).

### Example 32

### rac-N-(4-chlorobenzyl)-2-(((2-(4-fluorophenyl)-2-hydroxypropyl)oxy)methyl)-7-methyl-4-oxo-4,7-dihydrothieno[2,3-b]pyridine-5-carboxamide

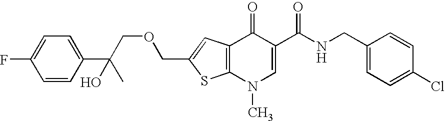

Physical characteristics. MS (El) m/z 514 (M⁺).

### Example 33

### rac-N-(4-chlorobenzyl)-2-(((2-(4-chlorophenyl)-2-hydroxypropyl)oxy)methyl)-7-methyl-4-oxo-4,7-dihydrothieno[2,3-b]pyridine-5-carboxamide

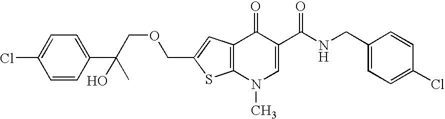

Physical characteristics. MS (El) m/z 530 (M⁺).

### Example 34

### rac-2-(((2-(4-bromophenyl)-2-hydroxypropyl)oxy)methyl)-N-(4-chlorobenzyl)-7-methyl-4-oxo-4,7-dihydrothieno[2,3-b]pyridine-5-carboxamide

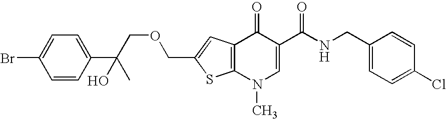

Physical characteristics. MS (El) m/z 574 (M⁺).

### Example 35

### rac-N-(4-chlorobenzyl)-2-(((2-(4-cyanophenyl)-2-hydroxypropyl)oxy)methyl)-7-methyl-4-oxo-4,7-dihydrothieno[2,3-b]pyridine-5-carboxamide

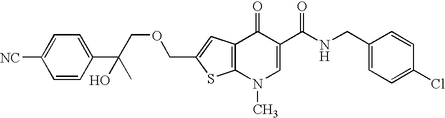

Physical characteristics. MS (El) m/z 521 (M⁺).

### Example 36

### N-(4-chlorobenzyl)-2-((((1R,2S)-2-hydroxy-1-methyl-2-phenylethyl)oxy)methyl)-7-methyl-4-oxo-4,7-dihydrothieno[2,3-b]pyridine-5-carboxamide

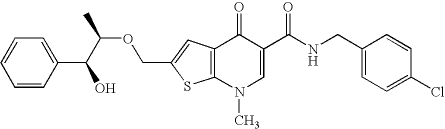

Physical characteristics. MS (El) m/z 496 (M⁺).

### Example 37

### N-(4-chlorobenzyl)-2-((((2R)-2-hydroxy-3-phenoxypropyl)oxy)methyl)-7-methyl-4-oxo-4,7-dihydrothieno[2,3-b]pyridine-5-carboxamide

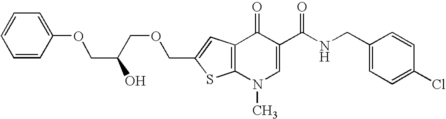

Physical characteristics. MS (El) m/z 512 (M⁺).

### Example 38

### N-(4-chlorobenzyl)-2-((((2R)-2-hydroxy-3-(2-methylphenoxy)propyl)oxy)methyl)-7-methyl-4-oxo-4,7-dihydrothieno[2,3-b]pyridine-5-carboxamide

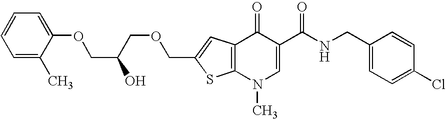

Physical characteristics. MS (El) m/z 516 (M⁺).

### Example 39

### N-(4-chlorobenzyl)-2-((((2R)-2-hydroxy-3-(2-methoxyphenoxy)propyl)oxy)methyl)-7-methyl-4-oxo-4,7-dihydrothieno[2,3-b]pyridine-5-carboxamide

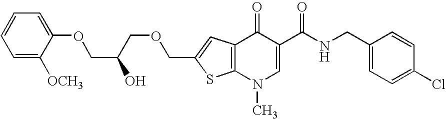

Physical characteristics. MS (El) m/z 542 (M⁺).

### Example 40

### N-(4-chlorobenzyl)-2-((((2R)-3-(4-chlorophenoxy)-2-hydroxypropyl)oxy)methyl)-7-methyl-4-oxo-4,7-dihydrothieno[2,3-b]pyridine-5-carboxamide

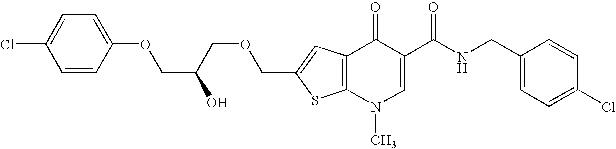

Physical characteristics. MS (El) m/z 546 (M⁺).

### Example 41

### N-(4-chlorobenzyl)-2-((((2R)-2-hydroxy-3-(1-naphthyloxy)propyl)oxy)methyl)-7-methyl-4-oxo-4,7-dihydrothieno[2,3-b]pyridine-5-carboxamide

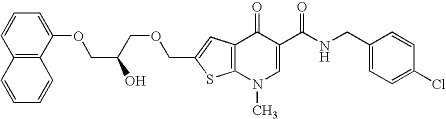

Physical characteristics. MS (El) m/z 562 (M⁺).

### Example 42

### N-(4-chlorobenzyl)-2-((((2R)-3-(2-chlorophenoxy)-2-hydroxypropyl)oxy)methyl)-7-methyl-4-oxo-4,7-dihydrothieno[2,3-b]pyridine-5-carboxamide

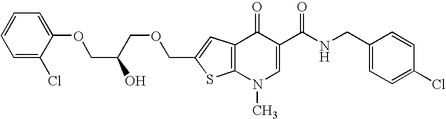

Physical characteristics. MS (El) m/z 546 (M⁺).

### Example 43

### N-(4-chlorobenzyl)-2-((((2R)-2-hydroxy-3(3-nitrophenoxy)propyl)oxy)methyl)-7-methyl-4-oxo-4,7-dihydrothieno[2,3-b]pyridine-5-carboxamide

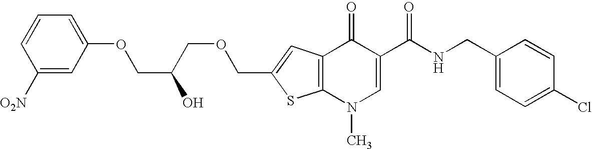

Physical characteristics. MS (El) m/z 557 (M⁺).

### Example 44

### N-(4-chlorobenzyl)-2-((((2R)-3-(2,3-dimethoxyphenoxy)-2-hydroxypropyl)oxy)methyl)-7-methyl-4-oxo-4,7-dihydrothieno[2,3-b]pyridine-5-carboxamide

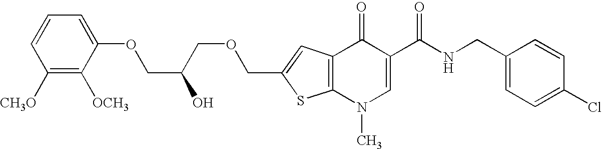

Physical characteristics. MS (El) m/z 572 (M⁺).

### Example 45

### N-(4-chlorobenzyl)-2-((((2R)-2-hydroxy-3-(4-methoxyphenoxy)propyl)oxy)methyl)-7-methyl-4-oxo-4,7-dihydrothieno[2,3-b]pyridine-5-carboxamide

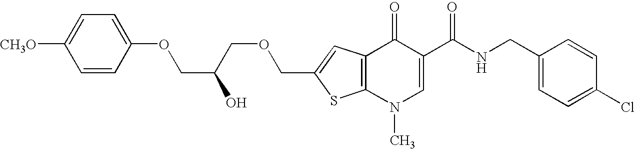

### Example 46

### N-(4-chlorobenzyl)-2-((((2R)-3-(4-fluorophenoxy)-2-hydroxypropyl)oxy)methyl)-7-methyl-4-oxo-4,7-dihydrothieno[2,3-b]pyridine-5-carboxamide

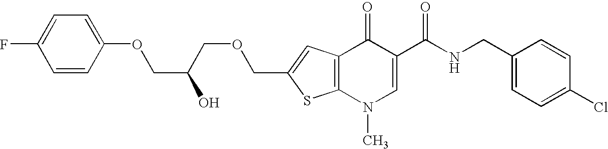

Physical characteristics. MS (El) m/z 530 (M⁺).

### Example 47

### rac-N-(4-chlorobenzyl)-2-(((2-hydroxy-3-(quinolin-8-yloxy)propyl)oxy)methyl)-7-methyl-4-oxo-4,7-dihydrothieno[2,3-b]pyridine-5-carboxamide

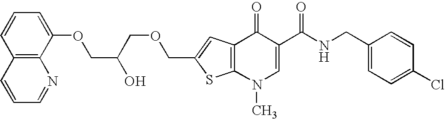

Physical characteristics. MS (El) m/z 563 (M⁺).

### Example 48

### N-(4-chlorobenzyl)-2-((((2R)-2-hydroxy-3-(pyridin-2-yloxy)propyl)oxy)methyl)-7-methyl-4-oxo-4,7-dihydrothieno[2,3-b]pyridine-5-carboxamide

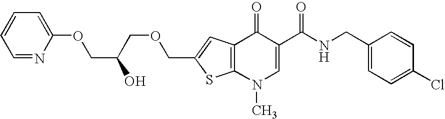

Physical characteristics. MS (El) m/z 513 (M⁺).

### Example 49

### N-(4-chlorobenzyl)-2-((((2R)-2-hydroxy-3-(pyridin-4-yloxy)propyl)oxy)methyl)-7-methyl-4-oxo-4,7-dihydrothieno[2,3-b]pyridine-5-carboxamide

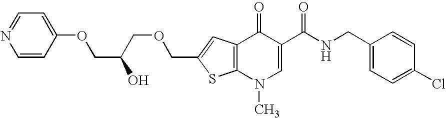

Physical characteristics. MS (El) m/z 513 (M⁺).

### Example 50

### N-(4-chlorobenzyl)-2-((((2R)-2-hydroxy-3-(pyrimidin-2-yloxy)propyl)oxy)methyl)-7-methyl-4-oxo-4,7-dihydrothieno[2,3-b]pyridine-5-carboxamide

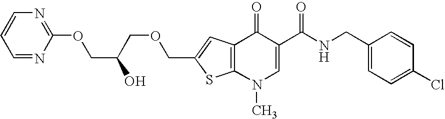

Physical characteristics. MS (El) m/z 514 (M⁺).

### Example 51

### N-(4-chlorobenzyl)-2-((((2R)-2-hydroxy-3-(pyrazin-2-yloxy)propyl)oxy)methyl)-7-methyl-4-oxo-4,7-dihydrothieno[2,3-b]pyridine-5-carboxamide

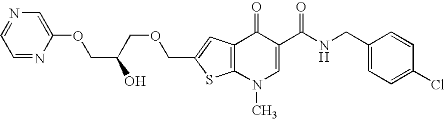

Physical characteristics. MS (El) m/z 514 (M⁺).

### Example 52

### N-(4-chlorobenzyl)-2-((((2R)-2-hydroxy-3-(1,3-thiazol-2-yloxy)propyl)oxy)methyl)-7-methyl-4-oxo-4,7-dihydrothieno[2,3-b]pyridine-5-carboxamide

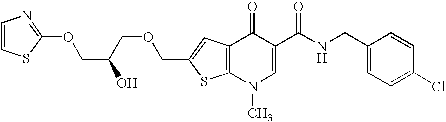

Physical characteristics. MS (El) m/z 519 (M⁺).

### Example 53

### N-(4-chlorobenzyl)-2-((((2R)-2-hydroxy-3-(1,3-thiazol-4-yloxy)propyl)oxy)methyl)-7-methyl-4-oxo-4,7-dihydrothieno[2,3-b]pyridine-5-carboxamide

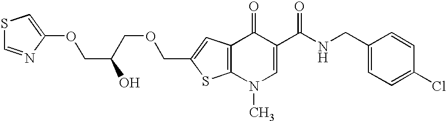

Physical characteristics. MS (El) m/z 519 (M⁺).

### Example 54

### N-(4-chlorobenzyl)-2-((((2R)-2-hydroxy-3-(i,3-thiazol-5-yloxy)propyl)oxy)methyl)-7-methyl-4-oxo-4,7-dihydrothieno[2,3-b]pyridine-5-carboxamide

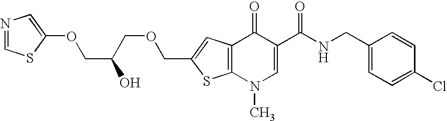

Physical characteristics. MS (El) m/z 519 (M⁺).

### Example 55

### rac-N-(4-chlorobenzyl)-2-((2-hydroxy-3-(methyl(phenyl)amino)propoxy)methyl)-7-methyl-4-oxo-4,7-dihydrothieno[2,3-b]pyridine-5-carboxamide

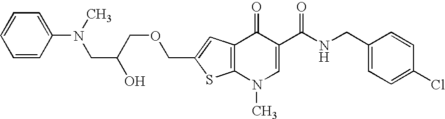

Physical characteristics. MS (El) m/z 525 (M⁺).

### Example 56

### rac-N-(4-chlorobenzyl)-2-((2-hydroxy-3-(ethyl(phenyl)amino)propoxy)methyl)-7-methyl-4-oxo-4,7-dihydrothieno[2,3-b]pyridine-5-carboxamide

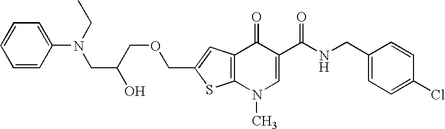

Physical characteristics. MS (El) m/z 539 (M⁺).

### Example 57

### N-(4-chlorobenzyl)-2-((((2R)-2-hydroxy-3-(phenylthio)propyl)oxy)methyl)-7-methyl-4-oxo-4,7-dihydrothieno[2,3-b]pyridine-5-carboxamide

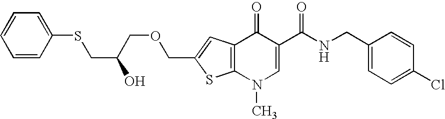

Physical characteristics. MS (El) m/z 528 (M⁺).

### Example 58

### N-(4-Chlorobenzyl)-2-((((3R)-3-hydroxy-3-phenylpropyl)oxy)methyl)-7-methyl-4-oxo-4,7-dihydrothieno[2,3-b]pyridine-5-carboxamide

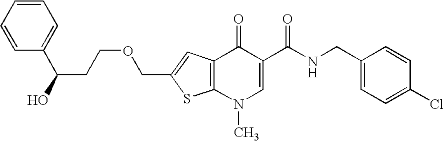

A mixture of (R)-1-phenyl-1,3-propanediol (100 mg) and dibutyltin oxide (164 mg) in toluene (3 mL) is heated to reflux for 2 h with removal of water by a Dean-Stark trap. The solvent is removed under vacuum and the residue was further dried (0.1 Torr, 2 h). Cesium fluoride (190 mg) was added followed by N-(4-chlorobenzyl)-2-(chloromethyl)-7-methyl-4-oxo-4,7-dihydrothieno[2,3-b]pyridine-5-carboxamide (Preparation 1, 419 mg) and DMF (5 mL). The suspension is stirred at room temperature for 72 h. The reaction mixture is poured into water (50 mL), extracted with CH₂Cl₂ (3×50 mL), and the combined organic layers are concentrated. The crude product is purified by column chromatography (CH₂Cl₂/methanol, 100/1; 50/1; 25/1) to afford 170 mg of the title compound as a white solid. Physical characteristics. M.p. 154.5-155.5° C.; ¹H NMR (400 MHz, DMSO-d₆) δ 10.57, 8.74, 7.42-7.20, 5.23, 4.71, 4.65, 4.55, 3.98, 3.60, 3.49, 1.85; ¹³C NMR (100 MHz, DMSO-d₆) δ 172.4, 164.7, 151.1, 146.3, 145.9, 138.9, 137.3, 131.7, 130.8, 129.5, 128.7, 128.4, 127.1, 126.1, 121.4, 114.8, 69.6, 67.2, 67.0, 43.3, 41.8, 39.5; MS (Cl) m/z 497 (MH⁺); Specific Rotation (25° C., D)=+13 (c 0.88, chloroform). Anal. Found: C, 62.73; H, 5.18; N, 5.63; Cl, 7.09; S, 6.42.

Employing procedures analogous to those described in Example 58 and utilizing the appropriate propane-1,3-diol, Examples 59-67 are prepared. The corresponding propane-1,3-diol are prepared according to literature procedures as follows where not commercially available. rac-1-(3-Methoxyphenyl)propane-1,3-diol (Example 60), rac-1-(3-chlorophenyl)propane-1,3-diol (Example 61), rac-1-(3-bromophenyl)propane-1,3-diol (Example 62), and rac-1-(3-fluorophenyl)propane-1,3-diol (Example 63) as described in Schaal, C. Bull Soc. Chim. Fr. 1973, 3083-3086. rac-1-(3-Methylphenyl)propane-1,3-diol (Example 59), rac-1-(4-methylphenyl)-propane-1,3-diol (Example 64), rac-1-(4-methoxyphenyl)propane-1,3-diol (Example 65), and rac-1-(4-bromophenyl)propane-1,3-diol (Example 66) as described in Yoshida, K.; Horikoshi, Y.; Eta, M.; Chikazawa, J.; Ogishima, M.; Fukuda, Y.; Sato, H. Bioorg. Med. Chem. Left. 1998, 8, 2967-2972. rac-1-(3,4-Dimethoxyphenyl)propane-1,3-diol (Example 67) as described in Pearl, I. A.; Gratzl, J. J. Org. Chem. 1962, 27, 2111-2114.

### Example 59

### rac-N-(4-chlorobenzyl)-2-((3-hydroxy-3-(3-methylphenyl)propoxy)methyl)-7-methyl-4-oxo-4,7-dihydrothieno[2,3-b]pyridine-5-carboxamide

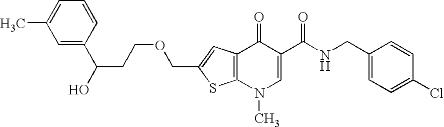

Physical characteristics. MS (El) m/z 510 (M⁺).

### Example 60

### rac-N-(4-chlorobenzyl)-2-((3-hydroxy-3-(3-methoxyphenyl)propoxy)methyl)-7-methyl-4-oxo-4,7-dihydrothieno[2,3-b]pyridine-5-carboxamide

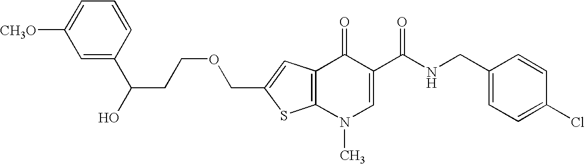

Physical characteristics. MS (El) m/z 526 (M⁺).

### Example 61

### rac-N-(4-chlorobenzyl)-2-((3-(3-chlorophenyl)-3-hydroxypropoxy)methyl)-7-methyl-4-oxo-4,7-dihydrothieno[2,3-b]pyridine-5-carboxamide

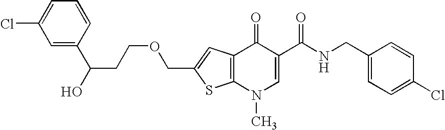

Physical characteristics. MS (El) m/z 530 (M⁺).

### Example 62

### rac-2-((3-(3-bromophenyl)-3-hydroxypropoxy)methyl)-N-(4-chlorobenzyl)-7-methyl-4-oxo-4,7-dihydrothieno[2,3-b]pyridine-5-carboxamide

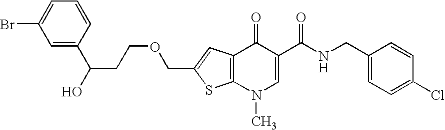

Physical characteristics. MS (El) m/z 574 (M⁺).

### Example 63

### rac-N-(4-chlorobenzyl)-2-((3-(3-fluorophenyl)-3-hydroxypropoxy)methyl)-7-methyl-4-oxo-4,7dihydrothieno[2,3-b]pyridine-5-carboxamide

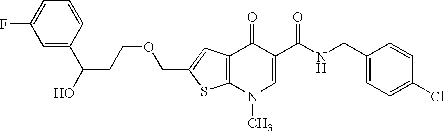

Physical characteristics. MS (El) m/z 514 (M⁺).

### Example 64

### rac-N-(4-chlorobenzyl)-2-((3-hydroxy-3-(4-methylphenyl)propoxy)methyl)-7-methyl-4-oxo-4,7-dihydrothieno[2,3-b]pyridine-5-carboxamide

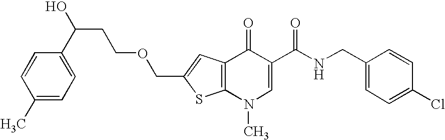

Physical characteristics. MS (El) m/z 510 (M⁺).

### Example 65

### rac-N-(4-chlorobenzyl)-2-((3-hydroxy-3-(4-methoxyphenyl)propoxy)methyl)-7-methyl-4-oxo-4,7-dihydrothieno[2,3-b]pyridine-5-carboxamide

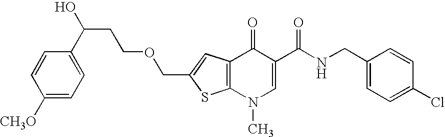

Physical characteristics. MS (El) m/z 526 (M⁺).

### Example 66

### rac-2-((3-(4-bromophenyl)-3-hydroxypropoxy)methyl)-N-(4-chlorobenzyl)-7-methyl-4-oxo-4,7-dihydrothieno[2,3-b]pyridine-5-carboxamide

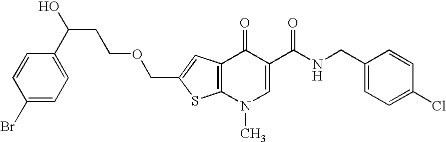

Physical characteristics. MS (El) m/z 574 (M⁺).

### Example 67

### rac-N-(4-chlorobenzyl)-2-((3-(3,4-dimethoxyphenyl)-3-hydroxypropoxy)methyl)-7-methyl-4-oxo-4,7-dihydrothieno[2,3-b]pyridine-5-carboxamide

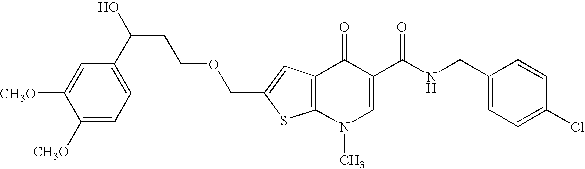

Physical characteristics. MS (El) m/z 556 (M⁺).

Preparation 2.

### N-(4-Chlorobenzyl)-7-ethyl-2-(hydroxymethyl)-4-oxo-4,7-dihydrothieno[2,3-b]pyridine-5-carboxamide

Potassium carbonate (0.87 g) and iodoethane (0.5 mL) are added to a solution of N-(4-chlorobenzyl)-4-hydroxy-2-(hydroxymethyl)thieno[2,3-b]pyridine-5-carboxamide (2.0 g, prepared as described in U.S. Pat. No. 6,239,142) in anhydrous DMF (60 mL). The reaction mixture is stirred at room temperature for 18 h. The mixture is diluted with water (150 mL) and filtered. The resulting white powder is washed with water (15 mL) followed by diethyl ether (15 mL) and dried in a vacuum oven to afford 1.64 g of the title compound as a white solid. Physical characteristics. Mp 169-172° C.; ¹H NMR (300 MHz, DMSO-d₆) δ 10.65, 8.74, 7.37, 7.29, 5.81, 4.70, 4.54, 4.32, 1.44. HRMS (FAB) m/z 377.0720 (M+H)⁺. Anal. Found: C, 56.87; H, 4.77; N, 7.38; Cl, 9.35; S, 8.44.

Preparation 3.

### N-(4-Chlorobenzyl)-2-(chloromethyl)-7-ethyl-4-oxo-4,7-dihydrothieno[2,3-b]pyridine-5-carboxamide

4-N,N-Dimethylaminopyridine (80 mg), 2,4,6-collidine (1.41 mL), and methanesulfonyl chloride (0.83 mL) are added to a solution of N-(4-chlorobenzyl)-7-ethyl-2-(hydroxymethyl)-4-oxo-4,7-dihydrothieno[2,3-b]pyridine-5-carboxamide (Preparation 2., 1.61 g) in anhydrous DMF (80 mL). The reaction mixture is stirred at room temperature for 24 h. The mixture is diluted with water (150 mL) and filtered. The resulting white powder is recrystallized from acetonitrile and dried in a vacuum oven to afford 1.4 g of the title compound as a white solid. Physical characteristics. Mp 199-200° C.; ¹H NMR (300 MHz, DMSO-d₆) δ 10.45, 8.77, 7.57, 7.38, 5.15, 4.54, 4.32, 1.44. Anal. Found: C, 54.53; H, 3.94; N, 7.03; Cl, 17.57; S, 8.09.

### Example 68

### N-(4-Chlorobenzyl)-7-ethyl-2-((((2S)-2-hydroxy-2-phenylethyl)oxy)methyl)-4-oxo-4,7-dihydrothieno[2,3-b]pyridine-5-carboxamide

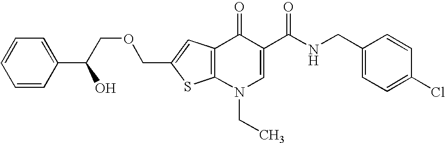

Employing the conditions described in Example 58, N-(4-chlorobenzyl)-2-(chloromethyl)-7-ethyl-4-oxo-4,7-dihydrothieno[2,3-b]pyridine-5-carboxamide (Preparation 3) is coupled with (S)-1-phenylethane-1,2-diol to afford the title compound.

Preparation 4.

### N-(4-Chlorobenzyl)-7-propyl-2-(hydroxymethyl)-4-oxo-4,7-dihydrothieno[2,3-b]pyridine-5-carboxamide

Potassium carbonate (0.91 g) and 1-iodopropane (0.64 mL) are added to a solution of N-(4-chlorobenzyl)-4-hydroxy-2-(hydroxymethyl)thieno[2,3-b]pyridine-5-carboxamide (2.0 g, prepared as described in U.S. Pat. No. 6,239,142) in anhydrous DMF (60 mL). The reaction mixture is stirred at room temperature for 4 h. The mixture is diluted with water (150 mL) and filtered. The resulting white powder is washed with water (15 mL) followed by diethyl ether (15 mL) and dried in a vacuum oven to afford 1.73 g of the title compound as a white solid. Physical characteristics. Mp 174-175° C. ¹H NMR (300 MHz, DMSO-d₆) δ 810.62, 8.72, 7.38, 7.29, 5.80, 4.69, 4.55, 4.27, 1.87, 0.89; Anal. Found: C, 58.20; H, 4.96; N, 7.13; Cl, 8.98; S, 8.16.

Preparation 5.

### N-(4-Chlorobenzyl)-2-(chloromethyl)-7-propyl-4-oxo-4,7-dihydrothieno[2,3-b]pyridine-5-carboxamide

4-N,N-Dimethylaminopyridine (80 mg), 2,4,6-collidine (1.39 mL), and methanesulfonyl chloride (0.81 mL) are added to a solution of N-(4-chlorobenzyl)-7-propyl-2-(hydroxymethyl)-4-oxo-4,7-dihydrothieno[2,3-b]pyridine-5-carboxamide (Preparation 4,1.63 g) in anhydrous DMF (80 mL). The reaction mixture is stirred at room temperature for 24 h. The mixture is diluted with water (150 mL) and filtered. The resulting light yellow powder is recrystallized from acetonitrile and dried in a vacuum oven to afford 1.4 g of the title compound as a light yellow solid. Physical characteristics. Mp 186.5-188° C.; ¹H NMR (300MHz, DMSO-d₆) δ 10.45, 8.75, 7.56, 7.39, 5.15, 4.54, 4.27, 1.85, 0.91. Anal. Found: C, 55.76; H, 4.59; N. 6.95; Cl, 16.88; S, 7.80.

### Example 69

### N-(4-Chlorobenzyl)-2-((((2S)-2-hydroxy-2-phenylethyl)oxy)methyl)-4-oxo-7-propyl-4,7-dihydrothieno[2,3-b]pyridine-5-carboxamide

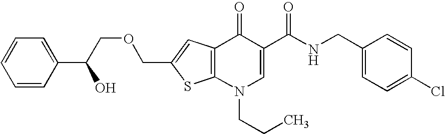

Employing the conditions described in Example 58, N-(4-chlorobenzyl)-2-(chloromethyl)-7-propyl-4-oxo-4,7-dihydrothieno[2,3-b]pyridine-5-carboxamide (Preparation 5) is coupled with (S)-1-phenylethane-1,2-diol to afford the title compound.

Preparation 6.

### N-(4-Chlorobenzyl)-2-(hydroxymethyl)-7-(2-methoxyethyl)-4-oxo-4,7-dihydrothieno[2,3-b]pyridine-5-carboxamide

Potassium carbonate (5.0 g) and bromoethylmethyl ether (5.0 g) are added to a solution of N-(4-chlorobenzyl)-4-hydroxy-2-(hydroxymethyl)thieno[2,3-b]pyridine-5-carboxamide (11.4 g, prepared as described in U.S. Pat. No. 6,239,142) in anhydrous DMF (350 mL). The reaction mixture is stirred at room temperature for 18 h. The mixture is diluted with water (600 mL) and filtered. The resulting white powder is dried in a vacuum oven to afford 8.44 g of the title compound as a white solid. Physical characteristics. M.p. 193° C.; ¹H NMR (300 MHz, DMSO-d6) δ 10.58, 8.65, 7.37, 7.29, 5.82, 4.70, 4.54, 4.47, 3.76, 3.24. HRMS (FAB) m/z 407.0836 (M+H)⁺. Anal. Found: C, 55.81; H, 4.71; N, 6.90; Cl, 8.58; S, 7.81.

Preparation 7.

### N-(4-Chlorobenzyl)-2-(chloromethyl)7-(2-methoxyethyl-4-oxo-4,7-dihydrothieno[2,3-b]pyridine-5-carboxamide

4-N,N-Dimethylaminopyridine (360 mg), 2,4,6-collidine (6.5 mL), and methanesulfonyl chloride (3.8 mL) are added to a solution of N-(4-chlorobenzyl)-2-(hydroxymethyl)-7-(2-methoxyethyl)-4-oxo-4,7-dihydrothieno[2,3-b]pyridine-carboxamide (Preparation 6, 8.0 g) in anhydrous DMF (360 mL). The reaction mixture is stirred at room temperature for 18 h. The mixture is diluted with water (600 mL) and filtered. The resulting off-white powder is dried in a vacuum oven to afford 7.03 g of the title compound as an off-white solid. Physical characteristics. Mp 192-193° C.; ¹H NMR (300 MHz, DMSO-d₆) δ 10.48, 8.67, 7.55, 7.37, 5.14, 4.53, 4.46, 3.74, 3.24; HRMS (FAB) m/z 425.0480 (M+H)⁺. Anal. Found: C, 53.38; H, 4.37; N, 6.66; Cl, 15.77; S, 7.69.

### Example 70

### N-(4-Chlorobenzyl)-2-((((2S)-2-hydroxy-2-phenylethyl)oxy)methyl)-7-(2-methoxyethyl)-4-oxo-4,7-dihydrothieno[2,3-b]pyridine-5-carboxamide

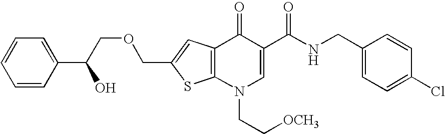

Employing the conditions described in Example 58, N-(4-chlorobenzyl)-2-(chloromethyl)-7-(2-methoxyethyl)-4-oxo-4,7-dihydrothieno[2,3-b]pyridine-5-carboxamide (Preparation 7) is coupled with (S)-1-phenylethane-1,2-diol to afford the title compound.

Preparation 8.

### N-(4-Chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide

Cesium carbonate (3.91 g) is added to a solution of N-(4-chlorobenzyl)-4-hydroxy-2-(hydroxymethyl)thieno[2,3-b]pyridine-5-carboxamide (3.49 g, prepared as described in U.S. Pat. No. 6,239,142) and 2-(2-iodoethoxy)tetrahydro-2H-pyran (2.56 g, prepared by mixing equal molar amounts of 2-iodoethanol and 3,4-dihydro-2H-pyran) in DMF (20 mL). The reaction mixture is stirred at 100° C. for 17 hours. The solvent is evaporated and the residue is dissolved in 10% CH₃OH in CH₂Cl₂. The mixture is washed with water and the organic layer is dried (MgSO₄), filtered, concentrated. The crude product is crystallized from EtOAc to afford 3.8 g of the title compound as a white solid. Physical characteristics. ¹H NMR (400 MHz, DMSO-d₆) δ 10.59, 8.71, 7.39, 7.38, 7.29, 5.79, 4.69, 4.58, 4.54, 4.48, 3.96, 3.78, 3.30, 1.54, 1.39, 1.29; MS (El) m/z 476 (M⁺); HRMS (FAB) m/z 477.1245 (M+H)⁺.

Preparation 9.

### N-(4-Chlorobenzyl)-2-(chloromethyl)-7-(2-hydroxyethyl)-4-oxo-4,7-dihydrothieno[2,3-b]pyridine-5-carboxamide

2,4,6-Collidine (2.9 mL) and a few crystals of 4-N,N-dimethylaminopyridine is added to a solution of N-(4-chlorobenzyl)-2-(hydroxymethyl)-4-oxo-7-(2-(tetrahydro-2H-pyran-2-yloxy)ethyl)-4,7-dihydrothieno[2,3-b]pyridine-5-carboxamide (Preparation 8., 3.5 g) in DMF (20 mL). Methanesulfonyl chloride (1.7 mL) is added drop-wise and the reaction mixture is stirred at room temperature for 72 h. The reaction mixture is poured into water (100 mL) and filtered. The solid is recrystallized from acetonitrile to afford 1.27 g of the title compound as a white solid. Physical characteristics. ¹H NMR (400 MHz, DMSO-d₆) δ 10.47, 8.67, 7.55, 7.40, 7.34, 5.15, 5.14, 4.54, 3.34, 2.51; MS (El ) m/z 410 (M⁺); HRMS (FAB) m/z 411.0332 (M+H)⁺. Anal. Found: C, 52.27; H, 4.05; N, 6.93.

### Example 71

### N-(4-Chlorobenzyl)-7-(2-hydroxyethyl)-2-((((2S)-2-hydroxy-2-phenylethyl)oxy)methyl)-4-oxo-4,7-dihydrothieno[2,3-b]pyridine-5-carboxamide

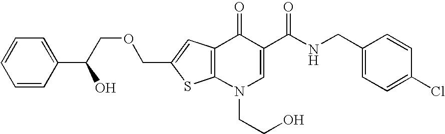

Employing the conditions described in Example 58, N-(4-chlorobenzyl)-2-(chloromethyl)-7-(2-hydroxyethyl)-4-oxo-4,7-dihydrothieno[2,3-b]pyridine-5-carboxamide (Preparation 9) is coupled with (S)-1-phenylethane-1,2-diol to afford the title compound.

Preparation 10.

### 4-Bromo-5-chloro-2-thiophenecarbaldehyde

n-Butyl lithium (2.5 M in hexanes, 105 mL) is slowly added to a solution of diisopropylamine (36.8 mL) in THF (600 mL) at 0° C. After 15 min, the mixture is cooled to −70° C. A solution of 3-bromo-2-chlorothiophene (49.4 g) in THF (20 mL) is added maintaining the internal temperature below −65° C. After 15 min, DMF (25.2 mL) is added. The mixture is stirred at −70° C. for 15 min and then allowed to warm to room temperature. The reaction mixture is quenched with saturated aq. NH₄Cl solution (200 mL) and concentrated in vacuo to one-half volume. The residue is diluted with EtOAc (500 mL) and the aqueous layer is separated. The aqueous layer is extracted with EtOAc (2×100 mL). The combined organic layers are washed with brine (100 mL), dried (MgSO₄), and concentrated to afford an oil. The oil is purified by column chromatography (heptane; heptane/EtOAc, 20/1; 10/1). After concentration the resulting solid is suspended in heptane (75 mL) and filtered to afford 30.3 g of the title compound as a light yellow solid. Physical characteristics. M.p.61-62° C.; ¹H NMR (300 MHz, DMSO-d₆) δ 89.82, 8.14; ¹³C NMR (100 MHz, CDCl₃) δ 181.6, 140.9, 138.5, 138.1, 112.8. Anal. Found: C, 26.68; H, 0.91; Br, 35.11; Cl, 15.78; S, 14.17.

Preparation 11.

### 4-((4-Bromo-5-chloro-2-thienyl)methyl)morpholine

Morpholine (15.2 mL), acetic acid (9.1 mL), and then sodium triacetoxyborohydride (50.3 g) is added to a solution of 4-bromo-5-chloro-2-thiophenecarbaldehyde (Preparation 10, 35.7 g) in 1,2-dichloroethane (600 mL) at 0° C. The mixture is allowed to warm to room temperature, and after 18 h, it is quenched with a 2N NaOH solution (200 mL) with ice bath cooling. The organic layer is separated and washed with a 1 N NaOH solution (2×200 mL). The combined aqueous layers are extracted with CH₂Cl₂ (2×100 mL). The combined organic layers are extracted with 0.25 M HCl solution (4×500 mL) and the resulting aqueous layer is made basic with 2N NaOH solution. The mixture is then extracted with CH₂Cl₂ (4×500 mL) and the organic layer is dried (Na₂SO₄) and concentrated to afford 39.24 g of the title compound as a light yellow oil. Physical characteristics. ¹H NMR (400 MHz, DMSO-d₆) δ 7.03, 3.63, 3.57, 2.42; 13C NMR (100 MHz, CDCl₃) δ 140.9, 127.3, 125.9, 109.2, 66.9, 57.6, 53.3; MS (ESl+) m/z 296 (M+H)⁺. Anal. Found: C, 36.49; H, 3.77; N, 4.70; Br, 26.73; Cl, 12.06; S,10.72.

Preparation 12.

### 1-(2-Chloro-5-(morpholin-4-ylmethyl)thien-3-yl)ethanone

4-((4-Bromo-5-chloro-2-thienyl)methyl)morpholine (Preparation 11, 44.5 g) is dissolved in diethyl ether (600 mL) and the solution is cooled to −70° C. n-Butyl lithium (2.5 M in hexanes, 66 mL) is added slowly maintaining the temperature below −65° C. The mixture is stirred at −70° C. for 15 min and then a solution of N-methoxy-N-methylacetamide (18.6 g) in diethyl ether (20 mL) is added maintaining the temperature below −60° C. After 10 min, the mixture is allowed to warm to room temperature and to stir for 18 h. The reaction mixture is then quenched with saturated aq. NH₄Cl (250 mL) followed by saturated aq. NaHCO₃ (200 mL). The mixture is extracted with EtOAc (4×200 mL). The combined organic layers are washed with brine (2×100 mL), dried (Na₂SO₄), and concentrated. The crude product is purified by column chromatography (heptane/acetone, 8/1) to afford 25.66 g of the title compound as a yellow oil. Physical characteristics. ¹H NMR (400 MHz, DMSO-d₆) δ 67.33, 3.63, 3.58, 2.51, 2.42; MS (ESl+) m/z 260 (M+H)⁺.

Preparation 13.

### Ethyl 3-(2-Chloro-5-(morpholin-4-ylmethyl)thien-3-yl)-3-oxopropanoate

Sodium hydride (60% dispersion in mineral oil, 16.7 g) is added to a cold (0° C.) solution of 1-(2-chloro-5-(morpholin-4-ylmethyl)thien-3-yl)ethanone (Preparation 12, 54.37 9) in diethylcarbonate (420 mL). The reaction mixture is allowed to warm to room temperature. After 1 h, the mixture is carefully warmed to 40° C. resulting in a vigorous exotherm causing the temperature to rise to 95° C. The mixture is allowed to cool to room temperature and is then quenched with glacial acetic acid (20 mL). The reaction mixture is diluted with water (350 mL) and saturated aq. Na₂CO₃ (200 mL), and the solution is extracted with MTBE (4×250 mL). The combined organic layers are washed with saturated aq. NaHCO₃ (50 mL) followed by brine (2×100 mL), dried (Na₂SO₄), and concentrated to provide an oil. The crude product is purified by column chromatography (heptane/acetone, 8/1; 5/1) to afford 51.7 g of the title compound as a yellow oil. Physical characteristics. ¹H NMR (400 MHz, DMSO-d₆) δ 12.45, 7.36, 7.25, 5.76, 4.22, 4.11, 4.04, 3.64, 3.58, 2.42, 1.26, 1.18; MS (ESl+) m/z 332 (M^{+H)}⁺.

Preparation 14.

### N-(4-Chlorobenzyl)-3-(2-chloro-5-(morpholin-4-ylmethyl)thien-3-yl)-3-oxopropanamide

A solution of ethyl 3-(2-chloro-5-(morpholin-4-ylmethyl)thien-3-yl)-3-oxopropanoate (Preparation 13, 51.7 g) in m-xylene (800 mL) is sparged with nitrogen gas for 15 min. 4-Chlorobenzylamine (20.0 mL) is added. The reaction mixture is heated to 140° C. for 2 h with collection of distillate in a Dean-Stark trap. The mixture is allowed to cool to room temperature and is then partially concentrated by rotary evaporation at 65° C. to provide a slurry. The slurry is suspended in diethyl ether (100 mL), filtered, and the resulting solids are washed with a mixture hexanes/diethyl ether (1/1,100 mL) to afford 48.96 g of the title compound as an off white solid. Physical characteristics. M.p. 109-112° C.; ¹H NMR (400 MHz, DMSO-d₆) δ 8.82, 8.64, 7.43-7.29, 7.11, 5.83, 4.38, 4.29, 3.85, 3.64, 3.62, 3.57, 2.41; ¹³C NMR (100 MHz, DMSO-d₆) δ 188.8, 172.0, 166.3, 162.8, 140.9 , 140.7, 138.6, 138.3, 135.5, 134.0, 131.9, 131.8, 131.7, 129.7, 129.4, 128.7, 128.6, 127.0, 126.6, 125.1, 92.4, 66.5, 56.9, 56.8, 53.2, 49.8, 41.9, 41.6. Anal. Found: C, 53.42; H, 4.66; N, 6.50; Cl, 16.53; S, 7.46.

Preparation 15.

### N-(4-Chlorobenzyl)-2-((2-chloro-5-(morpholin-4-ylmethyl)thien-3-yl)carbonyl)-3-((2-morpholin-ylethyl)amino)acrylamide

A mixture of N-(4-chlorobenzyl)-3-(2-chloro-5-(morpholin-4-ylmethyl)thien-3-yl)-3-oxopropanamide (Preparation 14, 20.0 g), triethylorthoformate (15.4 mL), and acetic anhydride (15.4 mL) is heated to 150° C. with removal of the distillate with a Dean-Stark trap. After 3 h, the volatiles are removed at 40 Torr (150° C.) and then at 0.2 Torr (100° C.) for 1 h to afford a brown oil. A portion of this residue (10.0 g) is dissolved in ethanol (150 mL) and N-aminoethylmorpholine (4.1 g) is added. The mixture is stirred at room temperature for 24 h and then is concentrated in vacuo. The crude product is purified by column chromatography (CH₂Cl₂/methanol, 9713) to afford 4.28 g of the title compound as a brown oil. Physical characteristics. ¹H NMR (300 MHz, DMSO-d₆) δ 10.51, 9.89, 7.62, 7.58, 7.37, 6.92, 4.44, 3.62, 3.58, 3.47, 3.40, 2.42, 2.32.

Preparation 16.

### N-(4-Chlorobenzyl)-7-(2-morpholin-4-ylethyl)-2-(morpholin-4-ylmethyl)-4-oxo-4,7-dihydrothieno[2,3-b]pyridine-5-carboxamide

A mixture of N-(4-chlorobenzyl)-2-((2-chloro-5-(morpholinylmethyl)thien-3-yl)carbonyl)-3-((2-morpholin-4-ylethyl)amino)acrylamide (Preparation 6,4.3 g) and K₂CO₃ (1.6 g) in DMF (75 mL) is stirred at 100° C. for 5 h. The mixture is allowed to cool to room temperature and is concentrated in vacuo. The crude product is triturated with EtOAc and filtered to afford 1.4 g of the title compound as a yellow solid. Physical characteristics. ¹H NMR (300 MHz, DMSO-d₆) δ 10.57, 8.71, 7.42-7.32, 4.55, 4.37, 3.74, 3.59, 3.50, 2.73, 2.44; MS (ESl+) m/z 531 (M+H)⁺.

Preparation 17.

### N-(4-Chlorobenzyl)-2-(chloromethyl)-7-(2-morpholin-4-ylethyl)-4-oxo-4,7-dihydrothieno[2,3-b]pyridine-5-carboxamide

N-(4-Chlorobenzyl)-7-(2-morpholin-4-ylethyl)-2-(morpholin-4-ylmethyl)-4-oxo-4,7-dihydrothieno[2,3-b]pyridine-5-carboxamide (Preparation 16, 1.3 g) is dissolved in chloroform (300 mL). Ethylchloroformate (0.58 mL) is added, and the mixture is stirred at room temperature for 18 h. Additional ethylchloroformate (0.58 mL) is added and stirring continued for 2 days. The mixture is concentrated in vacuo. The residue is triturated with ethyl acetate and filtered. The crude product is recrystallized from acetronitrile to afford 1.4 g of the title compound as a yellow solid. Physical characteristics. ¹H NMR (300 MHz, DMSO-d₆) δ 10.46, 8.75, 7.55, 7.42-7.32, 5.15,

### Example 72

### N-(4-Chlorobenzyl)-2-((((2S)-2-hydroxy-2-phenylethyl]oxy)methyl)-7-(2-morpholin-4-ylethyl)-4-oxo-4,7-dihydrothieno[2,3-b]pyridine-5-carboxamide

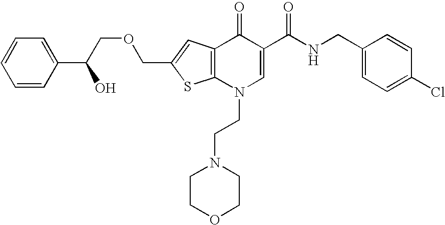

Employing the conditions described in Example 58, N-(4-Chlorobenzyl)-2-(chloromethyl)-7-(2-morpholin-4-ylethyl)-4-oxo-4,7-dihydrothieno[2,3-b]pyridine-5-carboxamide (Preparation 17) is coupled with (S)-1-phenylethane-1,2-diol to afford the title compound.

Preparation 18.

### Diethyl 2-(((3-(Ethoxycarbonyl)-4-methylthien-2-yl)amino)methylene)malonate

A mixture of ethyl 2-amino4-methylthiophene-3-carboxylate (5.23 g) and diethyl ethoxymethylenemalonate (5.71 mL) is heated to 135° C. for 3 h with a stream of nitrogen passing through the flask. The mixture is allowed to cool to room temperature. The resulting solid is recrystallized (EtOH, 250 mL) to afford 9.23 g of the title compound as yellow needles. Physical characteristics. M.p. 126-128° C.; ¹H NMR (400 MHz, DMSO-d₆) δ 12.36, 8.08, 6.77, 4.34, 4.24, 4.15, 2.31, 1.34, 1.27, 1.25; MS (ESl+) m/z 356 (M+H)⁺. Anal. Found: C, 54.12; H, 5.95; N. 3.99.; S. 8.99.

Preparation 19.

### 2-((3-Ethoxy-2-(ethoxycarbonyl)-3-oxoprop-1-enyl)amino)-4-methylthiophene-3-carboxylic acid

Potassium hydroxide (1.61 g) is dissolved in ethanol (10 mL) and heated to 70° C. A solution of diethyl 2-(((3-(ethoxycarbonyl)-4-methylthien-2-yl)amino)methylene)malonate (Preparation 18, 1.78 g) in THF (25 mL) is added. The resulting suspension is heated for an additional 2 h and is then allowed to cool to room temperature. The mixture is poured onto ice (250 mL) and the solution is made acidic with concentrated hydrochloric acid. The suspension is filtered, washed with 1 N hydrochloric acid, and dried (40 Torr, 75° C., 6 h) to afford 1.13 g of the title compound as a yellow solid. Physical characteristics. M.p. 190-192° C. (dec); ¹H NMR (400 MHz, DMSO-d₆) δ 13.38, 12.33, 8.08, 6.73, 4.23, 4.15, 2.30, 1.27, 1.24; MS (ESl−) m/z 326 (M−H)⁻.

Preparation 20.

### Ethyl 4-Hydroxy-3-methylthieno[2,3-b]pyridine-5-carboxylate

In a flask equipped with a Dean-Stark trap, a mixture of 2-((3-ethoxy-2-(ethoxycarbonyl)-3-oxoprop-1-enyl)amino)-4methylthiophene-3-carboxylic acid (Preparation 19, 4.58 g) and phenyl ether (50 mL) is degassed by freeze-pump-thaw method and then heated to reflux. After approximately 30 min, the reaction mixture is allowed to cool to room temperature. The crude mixture is purified by column chromatography (CH₂—Cl₂; CH₂—Cl₂ methanol, 100/1) to afford 1.95 g of the title compound as a yellow solid. Physical characteristics. M.p. 189-192° C.; ¹H NMR (400 MHz, CF₃CO₂D) δ 11.65, 9.10, 7.31, 4.66, 2.76, 1.53; MS (ESl−) m/z 236 (M−H)⁻. Anal. Found: C, 55.66; H, 4.67; N, 5.85; S, 13.40.

Preparation 21.

### Ethyl 4-Hydroxy-3-methyl-2-(morpholin-4-ylmethyl)thieno[2,3-b]pyridine-5-carboxylate

A suspension of ethyl 4-hydroxy-3-methylthieno[2,3-b]pyridine-5-carboxylate (Preparation 20, 0.92 g) and 4-methylenemorpholin-4-lum chloride (1.57 g) in acetonitrile (40 mL) is heated to 60° C. for 4 h. The resulting suspension is allowed to cool to room temperature, poured into water, and adjusted to neutral pH with saturated aq. sodium bicarbonate. The solution is extracted with CH₂—Cl₂ (3×100 mL). The combined organic layers are washed with brine (25 mL), dried (Na₂SO₄), and concentrated to afford 1.26 g of the title compound as a yellow solid. Physical characteristics. M.p. 182-185° C.; ¹H NMR (400 MHz, DMSO-d₆) δ 8.64, 4.37, 3.70, 3.38, 2.51, 2.47, 1.35; MS (ESl−) m/z 335 (M−H)⁻. Anal. Found: C, 56.93; H, 6.10; N, 8.26; S, 9.34.

Preparation 22.

### Ethyl 3,7-Dimethyl-2-(morpholin-4ylmethyl)-4-oxo-4,7-dihydrothieno[2,3-b]pyridine-5-carboxylate

Ethyl 4-hydroxy-3-methyl-2-(morpholin-4-ylmethyl)thieno[2,3-b]pyridine-5-carboxylate (Preparation 21, 1.47 g) and potassium carbonate (1.21 g) are suspended in DMF (25 mL). The mixture is warmed until the ester dissolves and iodomethane (0.30 mL) is added. The reaction mixture is allowed to stir at room temperature for 20 h. The mixture is then poured into water (100 mL) and extracted with EtOAc (3×100 mL). The combined organic layers are washed with brine (2×25 mL), dried (Na₂SO₄), and partially concentrated. Hexanes are added and the resulting solid is filtered to afford 0.70 g of the title compound as a light yellow solid. Physical characteristics. M.p. 172-173° C.; ¹H NMR (400 MHz, DMSO-d₆) δ 8.41, 4.19, 3.81, 3.65, 3.59, 2.48, 2.46, 1.27; MS (ESl+) m/z 351 (M+H)⁺. Anal. Found: C, 58.04; H, 6.54; N, 7.77; S. 8.97.

Preparation 23.

### N-(4-Chlorobenzyl)-3,7-dimethyl-2-(morpholin-4-ylmethyl)-4-oxo-4,7-dihydrothieno[2,3-b]pyridine-5-carboxamide

A mixture of ethyl 3,7-dimethyl-2-(morpholin-4-ylmethyl)-4-oxo-4,7-dihydrothieno[2,3-b]pyridine-5-carboxylate (Preparation 22, 700 mg) and 4-chlorobenzylamine (1.25 mL) is heated to 190° C. under a nitrogen atmosphere for 1 h. The reaction mixture is allowed to cool briefly and methanol (10 mL) is added. After cooling to room temperature, the product is filtered and washed with methanol and diethyl ether to afford 634 mg of the title compound as a white solid. Physical characteristics. M.p. 222-224° C.; ¹H NMR (400 MHz, DMSO-d₆) δ 10.67, 8.66, 7.41-7.33, 4.52, 3.92, 3.67, 3.59, 2.51, 2.47; MS (ESl+) m/z 446 (M+H)⁺. Anal. Found: C, 59.11; H, 5.47; N, 9.32; Cl, 7.96; S, 7.17.

Preparation 24.

### N-(4-Chlorobenzyl)-2-(chloromethyl)-3,7-dimethyl-4-oxo-4,7-dihydrothieno[2,3-b]pyridine-5-carboxamide

N-(4-Chlorobenzyl)-3,7-dimethyl-2-(morpholin-4-ylmethyl)-4-oxo-4,7-dihydrothieno[2,3-b]pyridine-5-carboxamide (Preparation 23, 500 mg) is dissolved in chloroform (10 mL) and ethyl chloroformate (268 μL) is added. The mixture is allowed to stir at room temperature for 24 h. The resulting suspension is diluted with diethyl ether (30 mL), filtered, and washed with diethyl ether to afford 415 mg of the title compound as a white solid. Physical characteristics. M.p. 234-238° C. (dec); ¹H NMR (400 MHz, DMSO-d₆) δ 10.55, 8.71, 7.41-7.39, 5.12, 4.53, 3.93, 2.58; MS (Cl) m/z 395 (M+H)⁺. Anal. Found: C, 54.36; H, 4.11; N, 6.99; Cl, 18.15; S, 7.94.

### Example 73

### N-(4-Chlorobenzyl)-3,7-dimethyl-2-((((2S)-2-hydroxy-2-phenylethyl)oxy)methyl)-4-oxo-4,7-dihydrothieno[2,3-b]pyridine-5-carboxamide

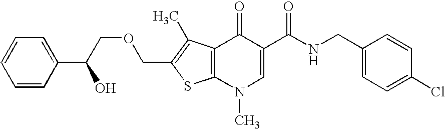

Employing the conditions described in Example 58, N-(4-chlorobenzyl)-2-(chloromethyl)-3,7-dimethyl-4-oxo-4,7-dihydrothieno[2,3-b]pyridine-5-carboxamide (Preparation 24) is coupled with (S)-1-phenylethane-1,2-diol to afford the title compound.

### Example 74

### N-(4-Chlorobenzyl)-7-methyl-4-oxo-2-((2-oxo-2-phenylethoxy)methyl)-4,7-dihydrothieno[2,3-b]pyridine-5-carboxamide

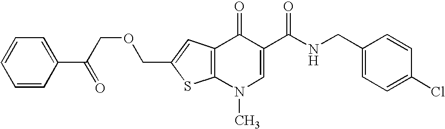

A mixture of N-(4-chlorobenzyl)-2-((2-hydroxy-2-phenylethoxy)methyl)-7-methyl-4-oxo-4,7-dihydrothieno[2,3-b]pyridine-5-carboxamide (Example 5, 425 mg) and Dess-Martin periodinane (450 mg) in CH₂Cl₂ (25 mL) is stirred at 20° C. for 18 hours. The mixture is filtered and the filtrate is purified by column chromatography (CH₂Cl₂/methanol, 98/2) to afford 356 mg of the title compound as a white solid. Physical characteristics. M.p. 209° C.; ¹H NMR (300 MHz, CDCl₃/CD₃OD) δ 8.70, 7.80-7.97, 7.53-7.60, 7.48, 7.37-7.47, 7.25, 4.86, 4.82, 4.56, 3.93.

### Example 75

### N-(4-Chlorobenzyl)-2-((2-(ethylamino)-2-phenylethoxy)methyl)-7-methyl-4-oxo-4,7-dihydrothieno[2,3-b]pyridine-5-carboxamide

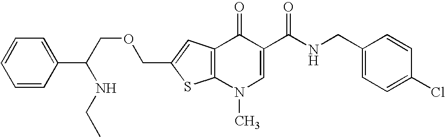

N-(4-Chlorobenzyl)-7-methyl-4-oxo-2-((2-oxo-2-phenylethoxy)methyl)-4,7-dihydrothieno[2,3-b]pyridine-5-carboxamide (Example 74, 290 mg) and ethylamine (350 μL) are dissolved in a solution of methanol/chloroform (5.0 mL, 1/2) and acetic acid is added to adjust the pH to 5. Molecular sieves (30 mg) are added. The reaction mixture is warmed to 50° C. and NaCNBH₄ (45 mg) is added. The reaction is stirred at 50° C. for 17 h, then poured into EtOAc (100 mL), washed with 1N NaOH and brine. The organic phase is dried (MgSO₄), filtered, and concentrated. The residue is purified by column chromatography (CH₂Cl₂/methanol, 95/5) and then recrystallized from methanoVEtOAc to afford 296 mg of the title compound as a white solid. Physical characteristics. ¹H NMR (300 MHz, CDCl₃) δ 10.57, 8.60, 7.43, 7.37-7.23, 5.60, 4.72, 4.62, 4.09, 3.89, 3.61, 2.50, 1.11. Anal. Found: C, 63.22; H, 5.54; N, 8.17.

### Example 76

### N-(4-Chlorobenzyl)-7-methyl-4-oxo-2-((2-phenyl-2-(propylamino)ethoxy)methyl)-4,7-dihydrothieno[2,3-b]pyridine-5-carboxamide

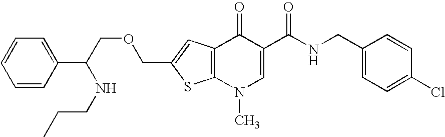

N-(4-Chlorobenzyl)-7-methyl-4-oxo-2-((2-oxo-2-phenylethoxy)methyl)-4,7-dihydrothieno[2,3-b]pyridine-5-carboxamide (Example 74, 150 mg) and propylamine (58 μL) are dissolved in a solution of methanol/chloroform (8.0 mL, 1/3) and acetic acid is added to adjust the pH to 5. Molecular sieves (30 mg) are added. The reaction is warmed to 50° C. for 1 h and NaCNBH₄ (31 mg) is added. The mixture is placed on a shaker block at 50° C. for 17 h, then poured into EtOAc (100 mL) and washed with 1N NaOH and brine. The organic layer is dried (MgSO₄), filtered, and concentrated. The residue is purified by column chromatography (CH₂Cl₂/methanol, 95/5) to afford 98 mg of the title compound as a white solid. Physical characteristics. ¹H NMR (400 MHz, DMSO-d₆) δ 10.6, 8.72,7.41-7.28, 7.23, 4.74, 4.53, 3.94, 3.89, 3.49, 2.27, 1.37, 0.81.

### Example 77

### N-(4-Chlorobenzyl)-2-((2-(dodecylamino)-2-phenylethoxy)methyl)-7-methyl-4-oxo-4,7-dihydrothieno[2,3-b]pyridine-5-carboxamide

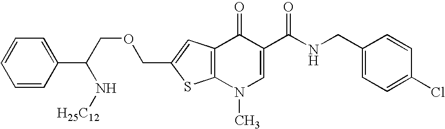

N-(4-Chlorobenzyl)-7-methyl-4-oxo-2-((2-oxo-2-phenylethoxy)methyl)-4,7-dihydrothieno[2,3-b]pyridine-5-carboxamide (Example 74, 150 mg) and dodecylamine (112 mg) are dissolved in a solution of methanol/chloroform (7.0 mL, 1/3) and acetic acid is added to adjust the pH to 5. Molecular sieves (30 mg) are added. The reaction is warmed to 50° C. for 1 h and then NaCNBH₄ (31 mg)is added. The mixture is placed on a shaker block at 50° C. for 17 h, then poured into EtOAc (100 mL) and washed with 1N NaOH and brine. The organic layer is dried (MgSO₄), filtered, and concentrated. The residue is purified by column chromatography (CH₂Cl₂/methanol, 95/5) to afford 123 mg of the title compound as a white solid. Physical characteristics. ¹H NMR (400 MHz, DMSO-d₆) δ 10.6, 8.72, 7.41-7.24, 4.74, 4.54, 3.95, 3.53, 3.49, 2.40, 1.35, 1.18, 0.84.

### Example 78

### N-(4-Chlorobenzyl)-2-((2-(cyclopropylamino)-2-phenylethoxy)methyl)-7-methyl-4-oxo-4,7-dihydrothieno[2,3-b]pyridine-5-carboxamide

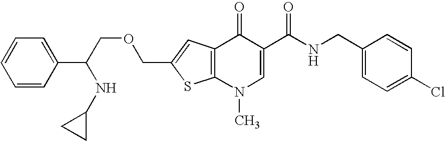

N-(4-Chlorobenzyl)-7-methyl-4-oxo-2-((2-oxo-2-phenylethoxy)methyl)-4,7-dihydrothieno[2,3-b]pyridine-5-carboxamide (Example 74, 150 mg) and cyclopropylamine (35 μL) are dissolved in chloroform/methanol (5 mL, 2/1) and the solution is made acidic with acetic acid. The reaction is stirred for 1 h at 55° C. Sodium cyanoborohydride (31 mg) is added and the reaction is placed on a shaker block at 55° C. for 18 h. The mixture is poured into CH₂Cl₂ (150 mL) and washed with 1 N sodium hydroxide, dried (MgSO₄), filtered, and concentrated. The residue is purified by column chromatography (CH₂Cl₂/methanol, 95/5) to afford 56 mg of the title compound as a white solid. Physical characteristics. ¹H NMR (400 MHz, DMSO-d₆) δ 10.6, 8.72, 7.41-7.22, 4.71, 4.54, 3.94, 3.88, 3.52, 3.23, 2.54, 1.89, 0.25, 0.19; HRMS (ESl) m/z 522. 1630.

### Example 79

### N-(4-Chlorobenzyl)-7-methyl-4-oxo-2-((2-phenyl-2-((pyridin-2-ylmethyl)amino)ethoxy)methyl)-4,7-dihydrothieno[2,3-b]pyridine-5-carboxamide

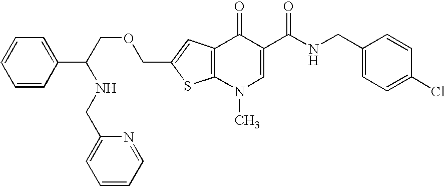

N-(4-Chlorobenzyl)-7-methyl-4-oxo-2-((2-oxo-2-phenylethoxy)methyl)-4,7-dihydrothieno[2,3-b]pyridine-5-carboxamide (Example 74, 60 mg) and pyridin-2-ylmethylamine (25 μL) are dissolved in chloroform/methanol (5 mL, 2/1) and the solution is made acidic with acetic acid. The reaction is stirred for 1 h at 55° C. Sodium cyanoborohydride (16 mg) is added and the reaction placed on a shaker block at 55° C. for 18 h. The mixture is poured into EtOAc (150 mL) and washed with 1N sodium hydroxide, dried (MgSO₄), filtered, and concentrated. The residue is purified by column chromatography (chloroform/methanol, 95/5) to afford 28 mg of the title compound as a white solid. Physical characteristics. ¹H NMR (400 MHz, DMSO-d₆) δ 10.55, 8.72, 8.49, 7.72, 7.41-7.25, 4.75, 4.54, 3.94, 3.91, 3.65, 3.54, 3.31; HRMS (ESl) m/z 573. 1716.

### Example 80

### rac-N-(4-Chlorobenzyl)-2-((2-hydroxy-1-phenylethoxy)methyl)-7-methyl-4-oxo-4,7-dihydrothieno[2,3-b]pyridine-5-carboxamide

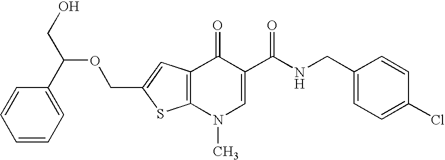

Employing conditions analogous to those in Example 5 and the same starting material, however, more extensive elution of the column affords the title compound as the more polar eluting isomer. Physical characteristics. ¹H NMR (400 MHz, DMSO-d₆) δ 10.56, 8.73, 7.45-7.27, 4.65, 4.54, 4.52-4.45, 3.97, 3.61, 3.48; HRMS (ESl) m/z 483. 1151.

1. A compound of general formula I 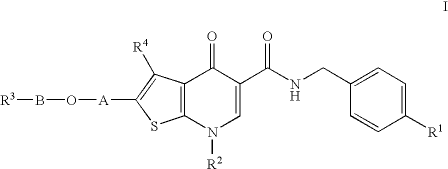
or a pharmaceutically acceptable salt thereof wherein
A is C₁₋₇ alkyl;
B is C₂₋₇ alkyl optionally substituted by one or more R⁵;
R¹ is
(a) Cl,
(b) Br,
(c) F,
(d) CN, or
(e) NO₂;
R² is
(a) (CH₂CH₂O)ᵢR⁶,
(b) het, wherein said het is bonded via a carbon atom,
(c) aryl,
(d) C₁₋₇alkyl which may be partially unsaturated and is optionally substituted by one or more R⁷ substituents, or
(e) C₃₋₈cycloalkyl which may be partially unsaturated and optionally substituted by one or more R⁷, or C₁₋₇alkyl optionally substituted by R⁷;
R³ is
(a) aryl,
(b) het,
(c) OR⁸,
(d) SR⁸,
(e) NR⁸R⁹,
(f) CH═N(OC₁₋₇alkyl), or
(g) ON═C(R⁶)₂;
R⁴ is
(a) H,
(b) halo, or
(c) C₁₋₄alkyl optionally substituted by halo;
R⁵ is
(a) OR⁶,
(b) SR⁶,
(c) NR⁹R⁹,
(d) halo,
(e) oxo, or
(f) phenyl optionally substituted by halo, C₁₋₇alkyl, or C₁₋₇alkoxy;
R⁶ is
(a) H, or
(b) C₁₋₇alkyl;
R⁷ is
(a) OR¹⁰,
(b) SR¹⁰,
(c) NR⁹R⁹,
(d) NR⁹(COR¹¹)
(e) halo,
(f) CONHR¹¹,
(g) CONR¹¹R¹¹,
(h) CO₂H,
(i) CO₂R¹¹,
(j) het,
(k) aryl,
(l) cyano,
(m) oxo, or
(n) SOₘR¹¹;
R⁸ is
(a) aryl, or
(b) het;
R⁹ is
(a) H,
(b) phenyl,
(c) C₃₋₈cycloalkyl, or
(d) C₁₋₁₆alkyl optionally substituted by OH, phenyl, pyridinyl, or halo;
R¹⁰ is
(a) H,
(b) aryl,
(c) het, wherein said het is bound through a carbon atom,
(d) C₁₋₇alkyl optionally substituted by aryl, het, OR⁶, SR⁶, NR⁶R⁶, halo, or C₃₋₈cycloalkyl optionally substituted by OR⁶, or
(e) C₃₋₈cycloalkyl optionally substituted by one or more substituents selected from halo, OR⁶ SR⁶, or NR⁶R⁶,
R¹¹ is
(a) aryl,
(b) het,
(c) C₃₋₈cycloalkyl, or
(d) C₁₋₇alkyl which may be partially unsaturated and is optionally substituted by one or more substituents selected from a group consisting of NR⁶R⁶, OR⁶, SR⁶, halo, het, or aryl;
each i is independently 2, 3, or 4;
each m is independently 1 or 2;
aryl is a phenyl radical optionally fused to a carbocyclic radical wherein aryl is optionally substituted with one or more R¹² substituents or any two adjacent R¹² substituents taken together constitute a group of the formula —O(CH₂)ₘO—;
het is a 4-16 membered saturated or unsaturated monocyclic, bicyclic, or tricyclic ring system having one (1), two (2), three (3) or four (4) heteroatoms selected from the group consisting of oxygen (—O—), sulfur (—S—), or nitrogen, wherein het is optionally substituted with one or more oxo (═O) or R¹² substituents;
R¹² is
(a) halo,
(b) OR¹³,
(c) SR⁶,
(d) NR⁶R⁶,
(e) phenyl, optionally substituted by halo, C₁₋₇alkyl, or C₁₋₇alkoxy,
(f) cyano,
(g) nitro,
(h) CONR⁶R⁶,
(i) CO₂R⁶,
(j) S(O)₂NR⁶R⁶,
(k) NR⁶(COR⁶),
(l) C₁₋₇alkyl which is optionally partially unsaturated and is optionally substituted by one or more R¹⁴, or
(m) C₃₋₈cycloalkyl;
R¹³ is
(a) H
(b) C₁₋₄alkyl optionally substituted by fluoro,
(c) phenyl optionally substituted by halo, C₁₋₇alkyl, or C₁₋₇alkoxy, or
(d) —(CH₂CH₂O)ₘR⁶;
and R¹⁴ is
(a) phenyl, optionally substituted by halo, C₁₋₇alkyl, or C₁₋₇alkoxy,
(b) OR⁶,
(c) SR⁶,
(d) NR⁶R⁶,
(e) 4-morpholine,
(f) CO₂R⁶,
(g) CONR⁶R⁶, or
(h) halo.

2. A compound of formula I as shown in claim 1 which is a formula IA:

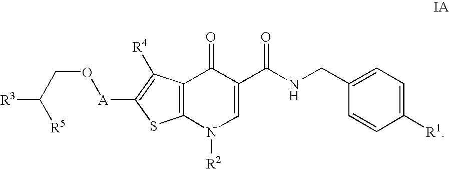

3. A compound of formula I in claim 1 which is a formula IB:

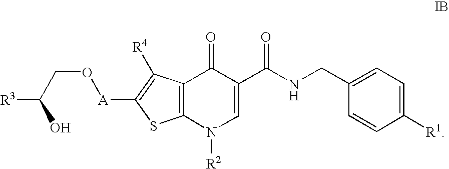

4. A compound of claim 1 wherein R¹ is Cl.

5. A compound of claim 1 wherein R² is C₁₋₄alkyl optionally substituted by one or more hydroxy, or C₁₋₄alkoxy.

6. A compound of claim 5 wherein R² is methyl.

7. A compound of claim 1 wherein R² is 2-hydroxyethyl, 3-hydroxypropyl, 2,3-dihydroxypropyl, or 5-hydroxypentyl.

8. A compound of claim 1 wherein R³ is phenyl optionally substituted with one to three halo, phenyl, OC₁₋₄alkyl, CN, C₁₋₄alkyl optionally substituted with one to three halo, NR⁶R⁶, CONR⁶R⁶, or CO₂R⁶; wherein R⁶ is H or C₁₋₄alkyl.

9. A compound of claim 1 wherein R³ is OR⁸ or SR⁸ wherein R⁸ is aryl.

10. A compound of claim 20 wherein R⁸ is aryl optionally substituted with one to three halo, phenyl, OC₁₋₄alkyl, CN, C₁₋₄alkyl optionally substituted with one to three halo, NR⁶R⁶, CONR⁶R⁶, or CO₂R⁶; wherein R⁶ is H or C₁₋₄alkyl.

11. A compound of claim 1 wherein R³ is NR⁸R⁹.

12. A compound of claim 1 which is
(1) N-(4-chlorobenzyl)-2-((((2S)-2-hydroxy-2-phenylethyl)oxy)methyl)-7-methyl-4-oxo-4,7-dihydrothieno[2,3-b]pyridine-5-carboxamide;
(2) 2-((((2S)-2-(1,1′-biphenyl-4-yl)-2-hydroxyethyl)oxy)methyl)-N-(4-chlorobenzyl)-7-methyl-4-oxo-4,7-dihydrothieno[2,3-b]pyridine-5-carboxamide;
(3) rac-N-(4-chlorobenzyl)-2-((2-hydroxy-3-(((1-methylethylidene)amino)-oxy)propoxy)methyl)-7-methyl-4-oxo-4,7-dihydrothieno[2,3-b]pyridine-5-carboxamide;
(4) rac-N-(4-chlorobenzyl)-2-(((3-(ethoxyimino)-2-hydroxypropyl)oxy)methyl)-7-methyl-4-oxo-4,7-dihydrothieno[2,3-b]pyridine-5-carboxamide;
(5) rac-N-(4-chlorobenzyl)-2-((2-hydroxy-2-phenylethoxy)methyl)-7-methyl-4-oxo-4,7-dihydrothieno[2,3-b]pyridine-5-carboxamide
(6) N-(4-chlorobenzyl)-2-((((2S)-2-(2-chlorophenyl)-2-hydroxyethyl)oxy)methyl)-7-methyl-4-oxo-4,7-dihydrothieno[2,3-b]pyridine-5-carboxamide;
(7) N-(4-chlorobenzyl)-2-((((2S)-2-hydroxy-2-(2-methoxyphenyl)ethyl)oxy)methyl)-7-methyl-4-oxo-4,7-dihydrothieno[2,3-b]pyridine-5-carboxamide;
(8) N-(4-chlorobenzyl)-2-((((2S)-2-hydroxy-2-(3-methoxyphenyl)ethyl)oxy)methyl)-7-methyl-4-oxo-4,7-dihydrothieno[2,3-b]pyridine-5-carboxamide;
(9) N-(4-chlorobenzyl)-2-((((2S)-2-hydroxy-2-(2-methylphenyl)ethyl)oxy)methyl)-7-methyl-4-oxo-4,7-dihydrothieno[2,3-b]pyridine-5-carboxamide;
(10) N-(4-chlorobenzyl)-2-((((2S)-2-hydroxy-2-(4-methylphenyl)ethyl)oxy)methyl)-7-methyl-4-oxo-4,7-dihydrothieno[2,3-b]pyridine-5-carboxamide;
(11) N-(4-chlorobenzyl)-2-((((2S)-2-hydroxy-2-(4-bromophenyl)ethyl)oxy)methyl)-7-methyl-4-oxo-4,7-dihydrothieno[2,3-b]pyridine-5-carboxamide;
(12) N-(4chlorobenzyl)-2-((((2S)-2-hydroxy-2-(2-naphthyl)ethyl)oxy)methyl)-7-methyl-4-oxo-4,7-dihydrothieno[2,3-b]pyridine-5-carboxamide;
(13) 2-((((2S)-2-((3,5-bis(trifluoromethyl)phenyl)-2-hydroxyethyl)oxy)methyl)-N-(4-chlorobenzyl)-7-methyl-4-oxo-4,7-dihydrothieno[2,3-b]pyridine-5-carboxamide;
(14) N-(4-chlorobenzyl)-2-((((2S)-2-(3-chlorophenyl)-2-hydroxyethyl)oxy)methyl)-7-methyl-4-oxo-4,7-dihydrothieno[2,3-b]pyridine-5-carboxamide;
(15) N-(4-chlorobenzyl)-2-((((2S)-2-4-chlorophenyl)-2-hydroethyl)oxy)methyl)-7-methyl-4-oxo-4,7-dihydrothieno[2,3-b]pyridine-5-carboxamide;
(16) N-(4-chlorobenzyl)-2-((((2S)-2-(4-fluorophenyl)-2-hydroxyethyl)oxy)methyl)-7-methyl-4-oxo-4,7-dihydrothieno[2,3-b]pyridine-5-carboxamide;
(17) N-(4-chlorobenzyl)-2-((((2S)-2-hydroxy-2-(4-(trifluoromethyl)phenyl)ethyl)oxy)methyl)-7-methyl-4-oxo-4,7-dihydrothieno[2,3-b]pyridine-5-carboxamide;
(18) N-(4-chlorobenzyl)-2-((((2S)-2-hydroxy-2-(4-methoxyphenyl)ethyl)oxy)methyl)-7-methyl-4-oxo-4,7-dihydrothieno[2,3-b]pyridine-5-carboxamide;
(19) N-(4-chlorobenzyl)-2-((((2S)-2-(3-cyanophenyl)-2-hydroxyethyl)oxy)methyl)-7-methyl-4-oxo-4,7-dihydrothieno[2,3-b]pyridine-5-carboxamide;
(20) 2-((((2S)-2-(3-(aminocarbonyl)phenyl)-2-hydroxyethyl)oxy)methyl)-N-(4-chlorobenzyl)-7-methyl-4-oxo-4,7-dihydrothieno[2,3-b]pyridine-5-carboxamide;
(21) methyl 4-((1S)-2-((5-(((4-chlorobenzyl)amino)carbonyl)-7-methyl-4-oxo-4,7-dihydrothieno[2,3-b]pyridin-2-yl)methoxy)-1-hydroxyethyl)benzoate;
(22) N-(4-chlorobenzyl)-2-((((2S)-2-(4-(dimethylamino)phenyl)-2-hydroxyethyl)oxy)methyl)-7-methyl-4-oxo-4,7-dihydrothieno[2,3-b]pyridine-5-carboxamide;
(23) 2-((((2S)-2-(3-bromo-4-methoxyphenyl)-2-hydroxyethyl)oxy)methyl)-N-(4-chlorobenzyl)-7-methyl-4-oxo-4,7-dihydrothieno[2,3-b]pyridine-5-carboxamide;
(24) N-(4-chlorobenzyl)-2-((((2S)-2-hydroxy-2-pyridin-2-ylethyl)oxy)methyl)-7-methyl-4-oxo-4,7-dihydrothieno[2,3-b]pyridine-5-carboxamide;
(25) N-(4-chlorobenzyl)-2-((((2S)-2-hydroxy-2-pyridin4-ylethyl)oxy)methyl)-7-methyl-4-oxo-4,7-dihydrothieno[2,3-b]pyridin-5-carboxamide;
(26) N-(4-chlorobenzyl)-2-((((2R)-2-(2-furyl)-2-hydroxyethyl)oxy)methyl)-7-methyl-4-oxo-4,7-dihydrothieno[2,3-b]pyridine-5-carboxamide;
(27) N-(4-chlorobenzyl)-2-((((2S)-2-hydroxy-2-phenylpropyl)oxy)methyl)-7-methyl-4-oxo-4,7-dihydrothieno[2,3-b]pyridine-5-carboxamide;
(28) N-(4-chlorobenzyl)-2-((((2S)-2-hydroxy-2-phenylbutyl)oxy)methyl)-7-methyl-4-oxo-4,7-dihydrothieno[2,3-b]pyridine-5-carboxamide;
(29) N-(4-chlorobenzyl)-2-((((2S)-2-hydroxy-3-methyl-2-phenylbutyl)oxy)methyl)-7-methyl-4-oxo-4,7-dihydrothieno[2,3-b]pyridine-5-carboxamide;
(30) N-(4-chlorobenzyl)-2-((((2S)-2-hydroxy-2,3-diphenylpropyl)oxy)methyl)-7-methyl-4-oxo-4,7-dihydrothieno[2,3-b]pyridine-5-carboxamide;
(31) rac-N-(4-chlorobenzyl)-2-(((2-hydroxy-3-methoxy-2-phenylpropyl)oxy)methyl)-7-methyl-4-oxo-4,7-dihydrothieno[2,3-b]pyridine-5-carboxamide;
(32) rac-N-(4-chlorobenzyl)-2-(((2-(4-fluorophenyl)-2-hydroxypropyl)oxy)methyl)-7-methyl-4-oxo-4,7-dihydrothieno[2,3-b]pyridine-5-carboxamide;
(33) rac-N-(4-chlorobenzyl)-2-(((2-(4-chlorophenyl)-2-hydroxypropyl)oxy)methyl)-7-methyl-4-oxo-4,7-dihydrothieno[2,3-b]pyridine-5-carboxamide;
(34) rac-2-(((2-(4-bromophenyl)-2-hydroxypropyl)oxy)methyl)-N-(4-chlorobenzyl-7-methyl-4-oxo-4,7-dihydrothieno[2,3-b]pyridine-5-carboxamide;
(35) rac-N-(4-chlorobenzyl)-2-(((2-(4-cyanophenyl)-2-hydroxypropyl)oxy)methyl)-7-methyl-4-oxo-4,7-dihydrothieno[2,3-b]pyridine-5-carboxamide;
(36) N-(4-chlorobenzyl)-2-((((1R,2S)-2-hydroxy-1-methyl-2-phenylethyl)oxy)methyl)-7-methyl-4-oxo-4,7-dihydrothieno[2,3-b]pyridine-5-carboxamide;
(37) N-(4chlorobenzyl)-2-((((2R)-2-hydroxy-3-phenoxypropyl)oxy)methyl)-7-methyl-4-oxo-4,7-dihydrothieno[2,3-b]pyridine-5-carboxamide;
(38) N-(4-chlorobenzyl)-2-((((2R)-2-hydroxy-3-(2-methylphenoxy)propyl)oxy)methyl)-7-methyl-4-oxo-4,7-dihydrothieno[2,3-b]pyridine-5-carboxamide;
(39) N-(4-chlorobenzyl)-2-((((2R)-2-hydroxy-3-(2-methoxyphenoxy)propyl)oxy)methyl)-7-methyl-4-oxo-4,7-dihydrothieno[2,3-b]pyridine-5-carboxamide;
(40) N-(4-chlorobenzyl)-2-((((2R)-3-(4-chlorophenoxy)-2-hydroxypropyl)oxy)methyl)-7-methyl-4-oxo-4,7-dihydrothieno[2,3-b]pyridine-5-carboxamide;
(41) N-(4-chlorobenzyl)-2-((((2R)-2-hydroxy-3-(1-naphthyloxy)propyl)oxy)methyl)-7-methyl-4-oxo-4,7-dihydrothieno[2,3-b]pyridine-5-carboxamide;
(42) N-(4-chlorobenzyl)-2-((((2R)-3-(2-chlorophenoxy)-2-hydroxypropyl)oxy)methyl)-7-methyl-4-oxo-4,7-dihydrothieno[2,3-b]pyridine-5-carboxamide;
(43) N-(4-chlorobenzyl)-2-((((2R)-2-hydroxy-3-(3-nitrophenoxy)propyl)oxy)methyl)-7-methyl-4-oxo-4,7-dihydrothieno[2,3-b]pyridine-5-carboxamide;
(44) N-(4-chlorobenzyl)-2-((((2R)-3-(2,3-dimethoxyphenoxy)-2-hydroxypropyl)oxy)methyl)-7-methyl-4-oxo-4,7-dihydrothieno[2,3-b]pyridine-5-carboxamide;
(45) N-(4-chlorobenzyl)-2-((((2R)-2-hydroxy-3-(4-methoxyphenoxy)propyl)oxy)methyl)-7-methyl-4-oxo-4,7-dihydrothieno[2,3-b]pyridine-5-carboxamide;
(46) N-(4-chlorobenzyl)-2-((((2R)-3-(4-fluorophenoxy)-2-hydroxypropyl)oxy)methyl)-7-methyl-4-oxo-4,7-dihydrothieno[2,3-b]pyridine-5-carboxamide;
(47) rac-N-(4-chlorobenzyl)-2-(((2-hydroxy-3-(quinolin-8-yloxy)propyl)oxy)methyl)-7-methyl-4-oxo-4,7-dihydrothieno[2,3-b]pyridine-5-carboxamide;
(48) N-(4-chlorobenzyl)-2-((((2R)-2-hydroxy-3-(pyridin-2-yloxy)propyl)oxy)methyl)-7-methyl-4-oxo-4,7-dihydrothieno[2,3-b]pyridine-5-carboxamide;
(49) N-(4-chlorobenzyl)-2-((((2R)-2-hydroxy-3-(pyridin-4-yloxy)propyl)oxy)methyl)-7-methyl-4-oxo-4,7-dihydrothieno[2,3-b]pyridine-5-carboxamide;
(50) N-(4-chlorobenzyl)-2-((((2R)-2-hydroxy-3-(pyrimidin-2-yloxy)propyl)oxy)methyl)-7-methyl-4-oxo-4,7-dihydrothieno[2,3-b]pyridine-5-carboxamide;
(51) N-(4-chlorobenzyl)-2-((((2R)-2-hydroxy-3-(pyrazin-2-yloxy)propyl)oxy)methyl)-7-methyl-4-oxo-4,7-dihydrothieno[2,3-b]pyridine-5-carboxamide;
(52) N-(4-chlorobenzyl)-2-((((2R)-2-hydroxy-3-(1,3-thiazol-2-yloxy)propyl)oxy)methyl)-7-methyl-4-oxo-4,7-dihydrothieno[2,3-b]pyridine-5-carboxamide;
(53) N-(4-chlorobenzyl)-2-((((2R)-2-hydroxy-3-(1,3-thiazol-4-yloxy)propyl)oxy)methyl)-7-methyl-4-oxo-4,7-dihydrothieno[2,3-b]pyridine-5-carboxamide;
(54) N-(4-chlorobenzyl)-2-((((2R)-2-hydroxy-3-(1,3-thiazol-5-yloxy)propyl)oxy)methyl)-7-methyl-4-oxo-4,7-dihydrothieno[2,3-b]pyridine-5-carboxamide;
(55) rac-N-(4-chlorobenzyl)-2-((2-hydroxy-3-(methyl(phenyl)amino)propoxy)methyl)-7-methyl-4-oxo-4,7-dihydrothieno[2,3-b]pyridine-5-carboxamide;
(56) rac-N-(4-chlorobenzyl)-2-((2-hydroxy-3-(ethyl(phenyl)amino)propoxy)methyl)-7-methyl-4-oxo-4,7-dihydrothieno[2,3-b]pyridine-5-carboxamide;
(57) N-(4-chlorobenzyl)-2-((((2R)-2-hydroxy-3-(phenylthio)propyl)oxy)methyl)-7-methyl-4-oxo-4,7-dihydrothieno[2,3-b]pyridine-5-carboxamide;
(58) N-(4-Chlorobenzyl)-2-((((3R)-3-hydroxy-3-phenylpropyl)oxy)methyl)-7-methyl-4-oxo-4,7-dihydrothieno[2,3-b]pyridine-5-carboxamide;
(59) rac-N-(4-chlorobenzyl)-2-((3-hydroxy-3-(3-methylphenyl)propoxy)methyl)-7-methyl-4-oxo-4,7-dihydrothieno[2,3-b]pyridine-5-carboxamide;
(60) rac-N-(4-chlorobenzyl)-2-((3-hydroxy-3-(3-methoxyphenyl)propoxy)methyl)-7-methyl-4-oxo-4,7-dihydrothieno[2,3-b]pyridine-5-carboxamide;
(61) rac-N-(4-chlorobenzyl)-2-((3-(3-chlorophenyl)-3-hydroxypropoxy)methyl)-7-methyl-4-oxo-4,7-dihydrothieno[2,3-b]pyridine-5-carboxamide;
(62) rac-2-((3-(3-bromophenyl)-3-hydroxypropoxy)methyl)-N-(4-chlorobenzyl)-7-methyl-4-oxo-4,7-dihydrothieno[2,3-b]pyridine-5-carboxamide;
(63) rac-N-(4-chlorobenzyl)-2-((3-(3-fluorophenyl)-3-hydroxypropoxy)methyl)-7-methyl-4-oxo-4,7-dihydrothieno[2,3-b]pyridine-5-carboxamide;
(64) rac-N-(4-chlorobenzyl)-2-((3-hydroxy-3-(4-methylphenyl)propoxy)methyl)-7-methyl-4-oxo-4,7-dihydrothieno[2,3-b]pyridine-5-carboxamide;
(65) rac-N-(4-chlorobenzyl)-2-((3-hydroxy-3-(4-methoxyphenyl)propoxy)methyl)-7-methyl-4-oxo-4,7-dihydrothieno[2,3-b]pyridine-5-carboxamide;
(66) rac-2-((3-(4-bromophenyl)-3-hydroxypropoxy)methyl)-N-(4-chlorobenzyl)-7-methyl-4-oxo-4,7-dihydrothieno[2,3-b]pyridine-5-carboxamide;
(67) rac-N-(4-chlorobenzyl)-2-((3-(3,4-dimethoxyphenyl)-3-hydroxypropoxy)methyl)-7-methyl-4-oxo-4,7-dihydrothieno[2,3-b]pyridine-5-carboxamide;
(68) N-(4-chlorobenzyl)-7-ethyl-2-((((2S)-2-hydroxy-2-phenylethyl)oxy)methyl)-4-oxo-4,7-dihydrothieno[2,3-b]pyridine-5-carboxamide;
(69) N-(4-chlorobenzyl)-2-((((2S)-2-hydroxy-2-phenylethyl)oxy)methyl)-4-oxo-7-propyl-4-7-dihydrothieno[2,3-b]pyridine-5-carboxamide;
(70) N-(4-chlorobenzyl)-2-((((2S)-2-hydroxy-2-phenylethyl)oxy)methyl)-7-(2-methoxyethyl)-4-oxo-4,7-dihydrothieno[2,3-b]pyridine-5-carboxamide;
(71) N-(4-chlorobenzyl)-7-(2-hydroxyethyl)-2-((((2S)-2-hydroxy-2-phenylethyl)oxy)methyl)-4-oxo-4,7-dihydrothieno[2,3-b]pyridine-5-carboxamide;
(72) N-(4-chlorobenzyl)-2-((((2S)-2-hydroxy-2-phenylethyl]oxy)methyl)-7-(2-morpholin-4-ylethyl)-4-oxo-4,7-dihydrothieno[2,3-b]pyridine-5-carboxamide;
(73) N-(4-chlorobenzyl)-3,7-dimethyl-2-((((2S)-2-hydroxy-2-phenylethyl)oxy)methyl)-4-oxo-4,7-dihydrothieno[2,3-b]pyridine-5-carboxamide;
(74) N-(4-chlorobenzyl)-7-methyl-4-oxo-2-((2-oxo-2-phenylethoxy)methyl)-4,7-dihydrothieno[2,3-b]pyridine-5-carboxamide;
(75) N-(4-chlorobenzyl)-2-((2-(ethylamino)-2-phenylethoxy)methyl)-7-methyl-4-oxo-4,7-dihydrothieno[2,3-b]pyridine-5-carboxamide;
(76) N-(4-chlorobenzyl)-7-methyl-4-oxo-2-((2-phenyl-2-(propylamino)ethoxy)methyl)-4,7-dihydrothieno[2,3-b]pyridine-5-carboxamide;
(77) N-(4-chlorobenzyl)-2-((2-(dodecylamino)-2-phenylethoxy)methyl)-7-methyl-4-oxo-4,7-dihydrothieno[2,3-b]pyridine-5-carboxamide;
(78) N-(4-chlorobenzyl)-2-((2-(cyclopropylamino)-2-phenylethoxy)methyl)-7-methyl-4-oxo-4,7-dihydrothieno[2,3-b]pyridine-5-carboxamide;
(79) N-(4-chlorobenzyl)-7-methyl-4-oxo-2-((2-phenyl-2-((pyridin-2-ylmethyl)-amino)ethoxy)methyl)-4,7-dihydrothieno[2,3-b]pyridine-5-carboxamide;
(80) rac-N-(4-chlorobenzyl)-2-((2-hydroxy-1-phenylethoxy)methyl)-7-methyl-4-oxo-4,7-dihydrothieno[2,3-b]pyridine-5-carboxamide;
or a pharmaceutically acceptable salt thereof.

13. A compound of claim 1 which is
(1) N-(4-chlorobenzyl)-2-((((2S)-2-hydroxy-2-phenylethyl)oxy)methyl)-7-methyl-4-oxo-4,7-dihydrothieno[2,3-b]pyridine-5-carboxamide;
(2) rac-N-(4-chlorobenzyl)-2-((2-hydroxy-2-phenylethoxy)methyl)-7-methyl-4-oxo-4,7-dihydrothieno[2,3-b]pyridine-5-carboxamide
(3) N-(4-Chlorobenzyl)-2-((((3R)-3-hydroxy-3-phenylpropyl)oxy)methyl)-7-methyl-4-oxo-4,7-dihydrothieno[2,3-b]pyridine-5-carboxamide;
(4) N-(4-Chlorobenzyl)-7-methyl-4-oxo-2-((2-oxo-2-phenylethoxy)methyl)-4,7-dihydrothieno[2,3-b]pyridine-5-carboxamide;
(5) N-(4-Chlorobenzyl)-2-((2-(ethylamino)-2-phenylethoxy)methyl)-7-methyl-4-oxo-4,7-dihydrothieno[2,3-b]pyridine-5-carboxamide;
(6) N-(4-Chlorobenzyl)-7-methyl-4-oxo-2-((2-phenyl-2-(propylamino)ethoxy)methyl)-4,7-dihydrothieno[2,3-b]pyridine-5-carboxamide;
(7) N-(4-Chlorobenzyl)-2-((2-(dodecylamino)-2-phenylethoxy)methyl)-7-methyl-4-oxo-4,7-dihydrothieno[2,3-b]pyridine-5-carboxamide;
(8) N-(4-chlorobenzyl)-2-((2-(cyclopropylamino)-2-phenylethoxy)methyl)-7-methyl-4-oxo-4,7-dihydrothieno[2,3-b]pyridine-5-carboxamide;
(9) N-(4-chlorobenzyl)-7-methyl-4-oxo-2-((2-phenyl-2-((pyridin-2-ylmethyl)-amino)ethoxy)methyl)-4,7-dihydrothieno[2,3-b]pyridine-5-carboxamide;
(10) rac-N-(4-chlorobenzyl)-2-((2-hydroxy-1-phenylethoxy)methyl)-7-methyl-4-oxo-4,7-dihydrothieno[2,3-b]pyridine-5-carboxamide;
or a pharmaceutically acceptable salt thereof.

14. A compound of claim 1 which is N-(4-chlorobenzyl)-2-((((2S)-2-hydroxy-2-phenylethyl)oxy)methyl)-7-methyl)oxo-4,7-dihydrothieno[2,3-b]pyridine-5-carboxamide.
